(19) United States
(12) Patent Application Publication
(10) Pub. No.: US 2024/0363258 A1
(43) Pub. Date: Oct. 31, 2024

(54) ZOONOTIC SPILL-OVER RISK ANALYTICS SYSTEM

(71) Applicant: Health Solutions Research, Inc., Rockville, MD (US)

(72) Inventors: Ajay Kumar Gupta, Potomac, MD (US); Ramani Peruvemba, McLean, VA (US)

(21) Appl. No.: 18/308,818

(22) Filed: Apr. 28, 2023

(51) Int. Cl. G16H 50/80

(52) U.S. Cl. CPC G16H 50/80

(57) ABSTRACT

A method and system for providing zoonotic spill-over risk analytics and/or prediction. The method includes obtaining data from at least one data source for at least one first locality of interest, removing source identification information from the data; and obtaining data on infection or disease incidence for at least one locality of interest. The method further includes training a model to identify an impact on the infection or disease incidence by determining which of the ecological, environmental, human migration, animal and insect migration, land use change, and climate change related factors (e.g., factors) impact the infection or disease incidence for the at least one locality of interest, and based on the factors having a highest impact on the infection or disease incidence, determine a zoonotic spill-over risk index for predicting a spill-over event. The model may be implemented with a second set of data to determine the zoonotic spill-over risk index for at least second locality of interest.

### FIELD OF THE DISCLOSURE

The present disclosure is generally related to systems and methods for providing zoonotic spill-over risk analytics and/or prediction; in one or more example embodiments, to identify an impact of ecological, environmental, human migration, animal and insect migration, land use change, and climate-change related factors on zoonotic spill-over events, and stratify a risk of the zoonotic spill-over event based on identified ecological, environmental, human migration, animal and insect migration, land use change, and climate-change related variables; and to identify the ecological, environmental, human migration, animal and insect migration, land use change, and climate-change related variables having the greatest impact on and/or prediction of the zoonotic spill-over event at specific localities of interest to prevent and/or mitigate the zoonotic spill-over event from becoming a pandemic.

### BACKGROUND

There are a number of different interconnected systems and international initiatives to develop technology and infrastructure for identifying and surveilling infectious diseases. Such systems, include gathering diseases data from all over the world to identify potentially dangerous pathogens in wildlife and determining whether the disease could seed the next pandemic, which include, e.g., Discovery & Exploration of Emerging Pathogens-Viral Zoonoses (DEEP VSN), or surveillance platforms for health data, including, Global Infectious Disease and Epidemiology Network (GIDEON), BioSense Platform, CommCare Architecture, DHIS2 Platform, Electronic Surveillance System for the Early Notification of Community-Based Epidemics (ESSENCE), MedShr Platform, Suite for Automated Global Electronic bioSurveillance (SAGES), or the like. There is no system or method presently available, however, that can provide a holistic approach for global surveillance that can identify specific ecological, environmental, human migration, animal and insect migration, land use change, and climate-change related variables that are predictive of zoonotic spill-over events for particular places or localities of interest to determine which localities of interest have the highest risk of a zoonotic spill-over event, and especially, a system and method that may be easily understandable and able to be displayed with the paired viewability from higher level to granular level to understand the effect of ecological, environmental, human migration, animal and insect migration, land use change, and climate-change related variables on the risk of zoonotic spill-over event, e.g., identify the degree to which specific ecological, environmental, human migration, animal and insect migration, land use change, and climate-change related factors impact the zoonotic spill-over event and the degree of improvement (e.g., reduction or prevention) of the zoonotic spill-over event from becoming a global pandemic should the specific ecological, environmental, human migration, animal and insect migration, land use change, and climate-change related variables be mitigated.

### SUMMARY

The present disclosure is generally related to systems and methods for providing zoonotic spill-over risk analytics and/or prediction; in one or more example embodiments, to identify an impact of ecological, environmental, human migration, animal and insect migration, land use change, and climate-change related factors on zoonotic spill-over events, and stratify a risk of the zoonotic spill-over event based on identified ecological, environmental, human migration, animal and insect migration, land use change, and climate-change related variables; and to identify the ecological, environmental, human migration, animal and insect migration, land use change, and climate-change related variables having the greatest impact on and/or prediction of the zoonotic spill-over event at specific localities of interest to prevent and/or mitigate the zoonotic spill-over event from becoming an epidemic or pandemic.

In at least one example embodiment, a method for providing zoonotic spill-over risk analytics and/or prediction is provided. The method includes obtaining a first set of ecological, environmental, human migration, animal and insect migration, land use change, and climate change related data from at least one data source for at least one first locality of interest, removing source identification information from the first set of ecological, environmental, human migration, animal and insect migration, land use change, and climate change related data, obtaining data on infection or disease incidence for the at least one locality of interest, and training a model. The training of the model includes identifying an impact of ecological, environmental, human migration, animal and insect migration, land use change, and climate change related variables on the infection or disease incidence by determining which of the ecological, environmental, human migration, animal and insect migration, land use change, and climate change related factors impact the infection or disease incidence for the at least one locality of interest, and based on the ecological, environmental, human migration, animal and insect migration, land use change, and climate change related variables having a highest impact on the infection or disease incidence, determine a zoonotic spill-over risk index for predicting a spill-over event for the at least one locality of interest. The method further includes implementing the model with a second set of ecological, environmental, human migration, animal and insect migration, land use change, and climate change related data from the at least one data source for at least a second locality of interest to determine the zoonotic spill-over risk index for the at least second locality of interest, and displaying at least one of the zoonotic spill-over risk index for the at least second locality of interest and the ecological, environmental, human migration, animal and insect migration, land use change, and climate change related variables having the highest impact on the zoonotic spill-over risk index for the at least second locality of interest.

In at least another example embodiment, a system for providing zoonotic spill-over risk analytics and/or prediction is provided. The system includes a plurality of data sources having ecological, environmental, human migration, animal and insect migration, land use change, and climate change related data and a cloud-based system configured to obtain a first set of the ecological, environmental, human migration, animal and insect migration, land use change, and climate change related data from the plurality of data sources. The cloud-based system includes machine-learning algorithms and is configured to remove source identification information from the first set of ecological, environmental, human migration, animal and insect migration, land use change, and climate change related data, obtain data on infection or disease incidence for the at least one locality of interest, identify an impact of ecological, environmental, human migration, animal and insect migration, land use change, and climate change related variables on the infection or disease incidence by determining which of the ecological, environmental, human migration, animal and insect migration, land use change, and climate change related factors impact the infection or disease incidence for the at least one locality of interest, and based on the ecological, environmental, human migration, animal and insect migration, land use change, and climate change related variables having a highest impact on the infection or disease incidence, determine a zoonotic spill-over risk index for predicting a spill-over event for the at least one locality of interest. The system is further configured to implement the model with a second set of ecological, environmental, human migration, animal and insect migration, land use change, and climate change related data from the at least one data source for at least a second locality of interest to determine the zoonotic spill-over risk index for the at least second locality of interest, and transmit at least one of the zoonotic spill-over risk index for the at least second locality of interest and the ecological, environmental, human migration, animal and insect migration, land use change, and climate change related variables having the highest impact on the zoonotic spill-over risk index for the at least second locality of interest.

### BRIEF DESCRIPTION OF THE DRAWINGS

The accompanying drawings illustrate various embodiments of systems, methods, and embodiments of various other aspects of the disclosure. Any person with ordinary skills in the art will appreciate that the illustrated element boundaries (e.g. boxes, groups of boxes, or other shapes) in the figures represent one example of the boundaries. It may be that in some examples one element may be designed as multiple elements or that multiple elements may be designed as one element. In some examples, an element shown as an internal component of one element may be implemented as an external component in another, and vice versa. Furthermore, elements may not be drawn to scale. Non-limiting and non-exhaustive descriptions are described with reference to the following drawings. The components in the figures are not necessarily to scale, emphasis instead being placed upon illustrating principles.

The present disclosure provides a detailed and specific description that refers to the accompanying drawings. The drawings and specific descriptions of the drawings, as well as any specific or alternative embodiments discussed, are intended to be read in conjunction with the entirety of this disclosure. The system and methods may be embodied in many different forms and should not be construed as being limited to the embodiments set forth herein; rather, these embodiments are provided by way of illustration only and so that this disclosure will be thorough, complete and fully convey understanding to those skilled in the art.

References are made to the accompanying drawings that form a part of this disclosure and which illustrate embodiments in which the systems and methods described in this specification may be practiced.

FIG. 1 is a schematic diagram illustrating a zoonotic spill-over event sequence, according to at least one example embodiment described herein.

FIG. 2 is a schematic diagram of a zoonotic spill-over risk analytics and/or prediction system, according to at least one example embodiment described herein.

FIG. 3 is a schematic diagram of a zoonotic spill-over risk analytics and/or prediction system, according to at least one other example embodiment described herein.

FIG. 4 is a schematic diagram of a zoonotic spill-over risk analytics and/or prediction system, according to at least another example embodiment described herein.

FIGS. 5, 6, and 7 are illustrated views of a dashboard for a zoonotic spill-over risk analytics and/or prediction system, according to at least another example embodiment described herein.

### DETAILED DESCRIPTION

The present disclosure is generally related to systems and methods for providing zoonotic spill-over risk analytics and/or prediction; in one or more example embodiments, to identify an impact of ecological, environmental, human migration, animal and insect migration, land use change, and climate-change related factors on zoonotic spill-over events, and stratify a risk of the zoonotic spill-over event based on identified ecological, environmental, human migration, animal and insect migration, land use change, and climate-change related variables; and to identify the ecological, environmental, human migration, animal and insect migration, land use change, and climate-change related variables having the greatest impact on and/or prediction of the zoonotic spill-over event at specific localities of interest to prevent and/or mitigate the zoonotic spill-over event from becoming a pandemic.

Embodiments of the present disclosure will be described more fully hereafter with reference to the accompanying drawings in which like numerals represent like elements throughout the several figures, and in which example embodiments are shown. Embodiments of the claims may, however, be embodied in many different forms and should not be construed as limited to the embodiments set forth herein. The examples set forth herein are non-limiting examples and are merely examples among other possible examples.

Some embodiments of this disclosure, illustrating all its features, will now be discussed in detail. The words “comprising,” “having,” “containing,” and “including,” and other forms thereof, are intended to be equivalent in meaning and be open ended in that an item or items following any one of these words is not meant to be an exhaustive listing of such item or items, or meant to be limited to only the listed item or items.

It must also be noted that as used herein and in the appended claims, the singular forms “a”, “an” and “the” include plural references unless the context clearly dictates otherwise. Although any systems and methods similar or equivalent to those described herein may be used in the practice or testing of embodiments of the present disclosure, the preferred, systems and methods are now described.

It is understood that a majority of the world's pandemics are the result of zoonotic spillover events, which may be when a disease within the animal population jumps to a human and is able to spread among humans, e.g., Ebola, Tuberculosis, HIV/AIDS, SARS-COV-1, and most recently SARS-COV-2, Monkeypox. It is appreciated that more than 6 out of every 10 known infectious disease have zoonotic origins, and 3 out of every 4 new or emerging infectious diseases in people may come from animals. Since such zoonosis may be novel to the human population, the human population may not have adequate immune protection to attack the zoonosis, which may increase the risk of spread of the zoonosis and severity of the same, e.g., resulting in a global pandemic.

While there are hundreds of potential pathogens, including, zoonosis, that humans are exposed to, rarely do such pathogens become global pandemics. However, it is understood that due to climate change, shifts in land use and food systems, deforestation, and economic development, e.g., urban expansion and human encroachment, the risk of such pathogens becoming global pandemics may be increased, at least due to the increased likelihood of human contact with wildlife, e.g., nonhuman animals.

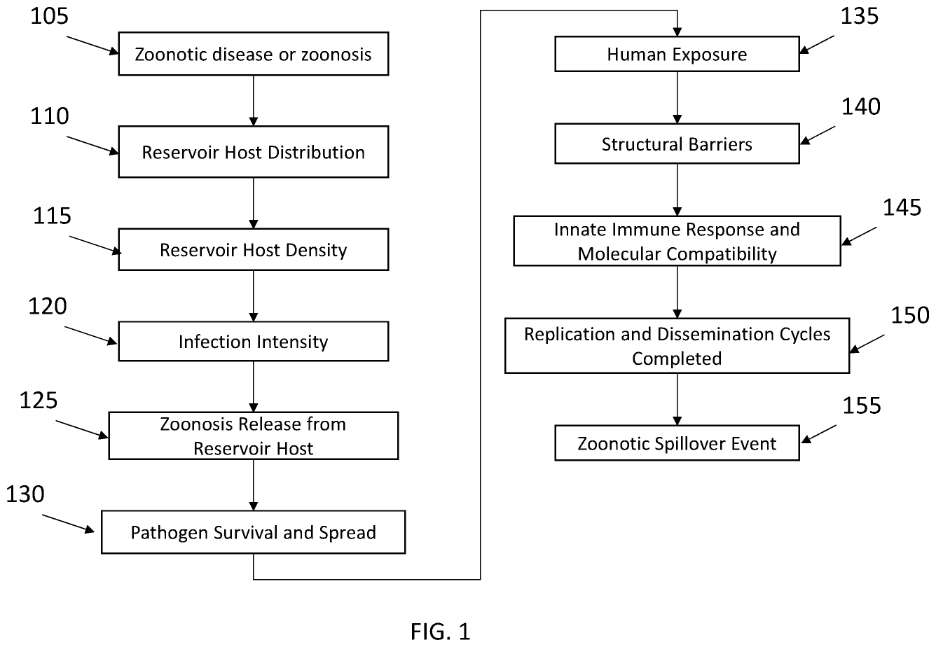

FIG. 1 illustrates an example embodiment of a pathway for a zoonotic spillover event. Initially, at 105, a zoonotic disease or zoonosis (or pathogen) is present that infects a host, e.g., a non-human animal, such as wildlife. At 110, the zoonosis may be distributed to a reservoir host in which the zoonosis may be maintained and from which the zoonosis may be transmitted. At 115, the zoonosis may spread to infect a density of the reservoir host, e.g., target non-human animal population, such as bats or monkeys. At 120, the zoonosis may increase in infection intensity such that more of the non-human animal population becomes infected. At 125, the zoonosis may be released from the reservoir host(s). If at, 130, the zoonosis survives and spreads, then at 135, humans may be exposed to the zoonosis. At 140, there may be structural barriers, such as, proximity to the non-human animal infected with the zoonosis that may reduce the likelihood of human exposure, but, as discussed above, due to climate change and/or urbanization and human encroachment, humans are becoming more and more in contact with non-human animals, e.g., wildlife. At 145, humans may have innate immune response and molecular non-compatibility. However, if at 145, humans do not have immune response and the zoonosis is molecularly compatible, at 150, the zoonosis may replicate and disseminate among the human population. If at 150, the zoonosis is disseminated among the human population, at 155, a zoonotic spillover event has occurred.

As such, systems and methods are described herein that are configured to determine factors, e.g., ecological, environmental, human migration, animal and insect migration, land use change, and climate change related factors, for predicting the risks of zoonotic spillover events occurring, for example, in different geographical locations, e.g., localities of interest, and/or determining when a zoonotic spillover event has occurred. In an embodiment, the systems and methods as described herein are directed to developing a more holistic understanding of the risk and risk factors for the prediction of zoonotic spillover events using advanced machine learning models that are trained on diverse datasets. In an embodiment, the advanced machine learning models are trained on a unique, fit-for-purpose infrastructure with adjustable components such that the overall infrastructure is specifically designed for the ingesting and analysis of the identified diverse datasets that are specifically relevant to zoonotic spillover threats and/or risk for the localities of interest. The infrastructure may also be configured to provide for the easier and quicker visualization and consumption of the risk of a zoonotic spill-over event occurring, threat identification of specific risk factors, as well as communication of both the specific risk factors (for a locality of interest) and/or action/response plans to mitigate the risk level for the zoonotic spill-over threat, e.g., by the WHO or other organization who may be involved with disease surveillance of the localit(ies) of interest. It is appreciated, while zoonotic events or zoonosis is described herein, the methods and systems as described herein may also be used to identify the factors and/or risks that predict other spillover events, including, but not limited to, fungal infections, parasitic infections, bacterial infections, or the like.

In at least one example embodiment, the methods and systems discussed herein may provide an index that shows or demonstrates the plain impact of various ecological, environmental, human migration, animal and insect migration, land use change, and climate change related factors on the zoonotic spill-over events and/or shows a predicted risk of a zoonotic spill-over event and/or to classify the same, e.g., severity of risk. In an embodiment, the index may be referred to as a Zoonotic Spill-Over Risk Index, which may assist decision makers to identify particular localities of interest that may be at the highest risk for a zoonotic spill-over event, e.g., areas of land or locations that may have emerging infectious diseases, to prevent and/or mitigate the zoonotic spill-over event from becoming a regional epidemic and/or a global pandemic. In an embodiment, the methods and systems may obtain and analyse a plurality, e.g., at least two, at least twelve, at least twenty, at least twenty-four, of different ecological, environmental, human migration, animal and insect migration, land use change, and climate-change related factors in determining the effects of the ecological, environmental, human migration, animal and insect migration, land use change, and climate-change related factors, and any combinations thereof, on the risk of a zoonotic spill-over event. The Zoonotic Spill-Over Risk Index may be determined using a machine learning model that includes an algorithm or model that may be used to determine weights and/or estimates of the plurality of different ecological, environmental, human migration, animal and insect migration, land use change, and climate-change related variables on the prediction of the zoonotic spill-over event. In an embodiment, the decisions makers may be public health decision makers, governmental decisions makers, global leaders, health system executives, public health analysts, insurance firms, pharmaceutical firms, and others who respond to health risks.

It will be appreciated that the “Zoonotic Spill-Over Risk Index” may refer to the magnitude or likelihood of a zoonotic spill-over event, both positively and negatively, based on the effect of the ecological, environmental, human migration, animal and insect migration, land use change, and climate change related variables on the zoonotic spill-over event. The scores/indices may be quantified (e.g., 1-5, 1-100, etc., with the higher the magnitude scores/indices indicating higher effect). For example, when the risk score is 3 or more over 5 (if the maximum score is 5) (or 60 or more over 100 (if the maximum score is 100)) for an area, the Zoonotic Spill-Over Risk Index may be an indication of a high risk or prediction of a zoonotic spill-over event occurring; when the risk score is less than 3 over 5 (or less than 60 over 100) for an area, the Zoonotic Spill-Over Risk Index may be an indication that there is a relatively lower risk or prediction that a zoonotic spill-over event may occur. Furthermore, the Zoonotic Spill-Over Risk Index may include scores/indices of the ecological, environmental, human migration, animal and insect migration, land use change, and climate change related variable in which, when the score/index is positive, the ecological, environmental, human migration, animal and insect migration, land use change, and climate-change related variable has a positive effect on the prediction of the risk of the zoonotic spill-over event, whereas, when the score/index is negative, the ecological, environmental, a human migration, animal and insect migration, land use change, and climate change related variable has a negative effect on the prediction of the risk of the zoonotic spill-over event.

In at least another example embodiment, the methods and systems may leverage a bi-variate cluster analysis or model to identify the specific ecological, environmental, human migration, animal and insect migration, land use change, and climate change related factors, e.g., variables, that impact the prediction and/or identification of risk of zoonotic spill-over events within specific regions as well as identified specific sub-regions to identify specific areas of higher risk. While the Zoonotic Spill-Over Risk Index may provide an index value for each of the ecological, environmental, human migration, animal and insect migration, land use change, and climate change related factors, the methods and systems may also leverage the determination of the index value for each of the ecological, environmental, human migration, animal and insect migration, land use change, and climate change related factors to provide decision makers a simple and actionable score/index at a granular level of the relative level of risk of a zoonotic spill-over event occurring and/or which specific ecological, environmental, human migration, animal and insect migration, land use change, and climate change related variables positively affect the prediction and/or occurrence of the zoonotic spill-over event.

As such, the zoonotic spill-over risk analytics and/or prediction system and method may be used to identify the risk of a zoonotic spill-over event occurring in a given geographical area, e.g., locality of interest, and make predictions that designate the most likely area for an emerging infectious disease. In an embodiment, the machine learning model may be configured to determine relative weight for each of the ecological, environmental, human migration, animal and insect migration, land use change, and climate change related factors and generate a heatmap of the risk of a zoonotic spill-over event occurring for various localities of interest that may be adjustable from higher granularity viewing to specific and focused localities of interest.

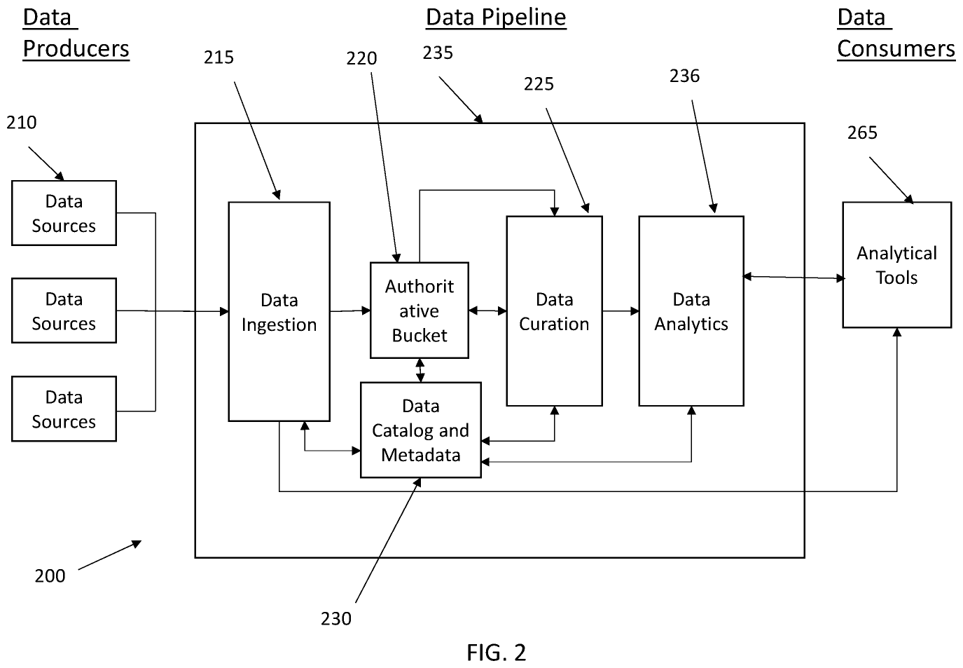

FIG. 2 illustrates a non-limiting example of a zoonotic spill-over risk analytics and/or prediction process 200 for identifying the impact of ecological, environmental, human migration, animal and insect migration, land use change, and climate change related factors on zoonotic spill-over events, e.g., infection/disease in human population, to determine the risk and/or likelihood of risk, e.g., prediction, of the ecological, environmental, human migration, animal and insect migration, land use change, and climate change related factors on the zoonotic spill-over event occurring. The zoonotic spill-over risk analytics and/or prediction process 200 may be implemented by a program, custom circuits, or by a combination thereof. The zoonotic spill-over risk analytics and/or prediction process 200 may include a data lake and serverless architecture for global scalability that achieves high velocity for near-real time processing and may include data producers, e.g., a plurality of data source(s) 210, data pipeline which may include a processing system 235 for ingesting, storing, analyzing, and/or transmitting the data, and may include a purpose built extract, transform and load dataflow pipeline, and data consumers/decision makers, e.g., end-users using analytical tools 265, such as, API or web-accessible servers to make decisions related to the risk of the zoonotic spill-over event occurring, e.g., public health decision makers, governmental decisions makers, health system executives, public health analysts, insurance firms, pharmaceutical firms, and others who respond to health risks.

The processing system 235 may be used to obtain a set of data from the plurality of data sources 210, including, but not limited to, ecological data, environmental data, climate change related data, and health data, which include, but are not limited to, Census data, foot traffic data/mobility data, human population and growth data, Earth Observation data from satellite imagery, including, but not limited to cloud cover, weather patterns, deforestation, vegetation, multispectral imaging, natural disasters, such as, volcanic eruptions, wildfires, and flooding, effects of climate change, or the like, global mammalian richness data, human mobility data, environmental data, genomic surveillance data, sampling data from animal populations, ground-level data, e.g., from road networks, Google Maps, or Open Street Maps, emerging and re-emerging infectious disease data, deforestation data, proximity of humans to wildlife data, rainfall, temperature, or other weather related dated, historical data on infection/disease incidence, historical data on symptomatology, and etc. from at least one data source(s) 210 for at least a particular locality of interest, e.g., a first locality of interest.

The various data source(s) 210 may be accessed and obtained by the processing system 235 by querying the data source(s) 210, for example, by including and using an ingestion/fetcher function 215. The ingestion function 215 may include a Lambda function, e.g., Python script, having a SQL search string to access and obtain a set of ecological, environmental, human migration, animal and insect migration, land use change, climate change related, and health data for the localities of interest from the data source(s) 210. In an embodiment, the ingestion function 215 may additionally include an extract, transform, and/or load service, e.g., Apache Kafka, Amazon Kinesis Firehouse, SageMaker Geospatial, or the like, to obtain and access the data source(s) 210 as streaming data and/or for enhancing or converting the geospatial data into numeric or tabular form. The data may be stored in an authoritative bucket 220, e.g., data lakes for storage on cloud-based and/or cloudless servers, after such processing and/or transformation.

The processing system 235 may further include a data processing and curation function 225 that may be configured to scrub data, e.g., metadata, from the data in the authoritative bucket 220. As such, the data from the data source(s) 210 may not include metadata, e.g., source identifying data, such that the data that is processed in the processing system 235 is secured and the data source may remain anonymous, e.g., source identifying information is removed. In an embodiment, the data processing and curation function 225 may be configured to convert the data into a risk signal, in which the underlying data is erased. For example, the data processing and curation function 225 may invoke an algorithm that is configured as a signal extraction function, e.g., trending of the data in a positive or negative direction, such that the risk of the factor may still be assessed, e.g., magnitude in the positive or negative direction, but the context of the data is removed, e.g., to provide security of the data. The data processing and curation function 225 may include algorithms written in object-oriented programming and functional programming scripts, e.g., Scala, Python, Node (Java), and/or accessed by open-source, distributed processing system, such as, Apache Spark, etc., for development of APIs in Java, Python, Scala, R, or the like.

In an embodiment, a data catalog and/or metadata function 230, e.g., a crawler function 230, may be used that is configured to obtain the data from the authoritative bucket 220 to populate the data for analysis by the processing system 235. The crawler function 230 may be configured to convert the data into tabular form in which any geospatial identification data may be removed. The crawler function 230 may be configured to crawl the data source(s) 210 in a single run such that the crawler function 230 may determine the format, schema, and associated properties in the raw data in the authoritative bucket 220, group the data into tables or partitions, and write or add data for analysis. In an embodiment, the crawler function 230 may include AWS SageMaker Geospatial or other image processing platform to access, interact, and interrogate imagery, including, but not limited to satellite imagery, and convert the imagery to numerical/tabular form.

The data processing and curation function 225 and/or the crawler function 230 may then be configured to populate the respective transformed data for analysis by a data analytics function 236 having memory to store the data, for example, in a hive data format.

The data analytics function 236 may include ML model(s) that may be used to generate a Zoonotic Spill-Over Risk Index which may be an indication of a prediction of an occurrence of a zoonotic spill-over event and/or which ecological, environmental, human migration, animal and insect migration, land use change, and climate change related variables have the highest impact on the prediction of the zoonotic spill-over event, e.g., based on infection or disease incidence and/or past historical indication of a zoonotic spill-over event.

In a non-limiting example, the data analytics function 236 may include a loop that includes a plurality of artificial intelligence based Machine-Learning Models that may be included in search functions, e.g., SQL search strings and/or Lambda functions for calling the ML/AI models, that is configured to access the hive data and perform at least the following functions/processing. In an embodiment, the data analytics function 236 may include at least one of an internal crawler function, an intermediate calculation function, and an intermediate data processing event function and may be connected to the data processing and curation function 225 and/or the data catalog and/or metadata function 230. In an embodiment, the data analytics function 236 may be configured to remove erroneous data, ensure the data has proper geocoding data and format, make necessary corrections to an imagery data obtained, e.g., atmospheric data correction of satellite imagery, for example, remove cloud cover, process any imagery data, e.g., using ENVI, ARTS software, or Amazon SageMaker Geospatial, to access and/or manipulate satellite imagery, e.g., from Sentinel and LandSAT, and/or stitch any imagery data together, label and identify the data, e.g., tagged data from the web or identify the data, e.g., as a wildfire, and/or train the ML/AI models.

In an embodiment, the data analytics function 236 may also include query search strings having ML-based models to identify an impact of the ecological, environmental, human migration, animal and insect migration, land use change, and climate change related variables on the infection or disease incidence that may be used to predict the occurrence of a zoological spill-over event for the particular locality of interest. For example, the ML model(s) may run an inference function in which the endpoint, e.g., infection or disease incidence or historical zoonotic spill-over event, is identified and the ecological, environmental, human migration, animal and insect migration, land use change, and climate change related factors in the data are identified to pass to the model, e.g., training using Amazon SageMaker and/or Amazon SageMaker Geospatial to deploy the various algorithms/functions. In embodiment, Amazon SageMaker Geospatial may be configured to enable access to base satellite imagery along with the ability to perform algebraic functions on raster data enabling the extraction of feature data from the satellite imagery and further ingesting the satellite imagery and extracted features for analysis; in addition, ML and Al processing of satellite data, geospatial data, and tabular data for, for example, land use land cover classification, analysis, and object detection. In an embodiment, the ML model(s) may be configured to perform at least a bi-variate cluster analysis to identify the ecological, environmental, human migration, animal and insect migration, land use change, and climate change related factors having the highest impact on the zoonotic spill-over event or the prediction of a zoonotic spill-over event, e.g., based on infection or disease incidence or historical data of a zoonotic spill-over event. In an embodiment, the bi-variate cluster analysis may be configured to identify features or factors in the data that are as similar as possible, and/or that are different as possible, including, but not limited to disease incidence and severity, at various levels of the localities of interest, e.g., global, national, state, or regional and ZIP Code, from epidemiology reports. The bi-variate cluster analysis may also include a varying coefficient model, weighted distance measuring model, K-means and hierarchical clustering models, or the like for processing the data. In an embodiment, the data may be gathered and analysed for the different levels of the localities of interest, e.g., national, regional, state, neighbourhood, such that the data is available or accessible for the different levels of the localities of interest.

In an embodiment, data analytics function 236 may be configured to include one or more of the following variables: ecological, environmental, human migration, animal and insect migration, land use change, and climate change related factors determined from the Journal of Infectious Disease (controlling for spatial and temporal bias), human population growth, emerging and infectious disease events, indicator(s) of rapid human population growth, wildlife host species richness, mobility data, deforestation data, proximity of humans to wildlife, sampling/testing data, e.g., from DEEP VZN, average rainfall, and the like. The data analytics function 236 may then use a ML model that includes a logistic regression of the variables, such as the following:

$$log ( pi / 1 - pi ) ( EID ) = B 0 + B ( JID ) + B ( Population Growth ) + B ( EID ) + B ( Indicator Population Growth ) + B ( Host Species Richness ) + B ( Mob Data ) + B ( Deforst ) + B ( Sample / Test ) + B ( Prox to Wildlife ) + B ( Avg Rainfall )$$

The logistic regression may also include ML models that further include historical rates of infection and symptomology for a plurality of diseases. To the infections/disease incidence and historical data on disease symptomology can be applied clustering and classification algorithms to detect and understand where potentially anomalous clusters of symptoms are occurring.

As such, based in the ecological, environmental, human migration, animal and insect migration, land use change, climate change related data, and historical infection or disease incidence, the ecological, environmental, human migration, animal and insect migration, land use change, and climate change related variables having the highest impact on the indication and/or prediction of the zoonotic spill-over event may be identified and quantified, e.g., iteratively learning from prior Zoonotic Spill-Over Risk Index determinations and/or prior infections, diseases, and zoonotic spill-over events on which variables have the greatest impact, both positively and negatively, on the prediction of a zoonotic spill-over event. In an embodiment, the top five, ten, twenty, thirty, forty, or fifty weighted variables may be selected for the ML model(s). Moreover, the data analytics function 236 may include trained ML models that may be deployed for inference that predict future zoonotic spill-over events, e.g., future infection incidences based on a second set of ecological, environmental, human migration, animal and insect migration, land use change, and climate change related data.

In an embodiment, the ML models and/or algorithms may be trained as follows. A set of data, including, but not limited to ecological, environmental, human migration, animal and insect migration, land use change, climate change related data and historical disease and symptomatology may be obtained, e.g., via download or through APIs, from a diverse range of sources for anomaly detection, e.g., from the data sources 210. In an embodiment, population data and clinical data, e.g., disease incidence and severity, may be used since personal identifying information does not have been captured. From the disease incidence and population data, prevalence of the zoonosis may be determined and from the severity and underlying health risk data, mortality risk may be determined. That is, to the infections/disease incidence and historical data on disease symptomology can be applied clustering and classification algorithms to detect and understand where potentially anomalous clusters of symptoms are occurring.

After the data has been collected and loaded into the authoritative bucket 220, a plurality of lambda functions which contain script for data processing and transformation may be triggered for processing the data in the authoritative bucket 220. In an embodiment, the lambda functions may include, Athena, Glue Crawlers, Python, NumPy, pandas, and other related packages to process and transform the data into tabular form suitable for training the ML models.

Based on the collected data, the data analytics function 236 may be used to generate and/or train a ML model for determining the Zoonotic Spill-Over Risk Index which may be an indication of which ecological, environmental, human migration, animal and insect migration, land use change, and climate change related factors have the highest impact on the prediction of an occurrence of a zoonotic spill-over event. That is, the training data may be fed into a regression classification algorithm, e.g., ML model, to determine relative weights of each of the ecological, environmental, human migration, animal and insect migration, land use change, and climate change related factors to be included in the ML model. The ML model may then be trained using the clinical level data to predict disease incidence based on the historical symptomatology and disease data using clustering and classification algorithms to determine whether zoonotic spill-over events have occurred or whether potential zoonotic spill-over events have occurred. For example, to the infections/disease incidence and historical data on disease symptomology can be applied clustering and classification algorithms to detect and understand where potentially anomalous clusters of symptoms are occurring.

In an embodiment, for the classification and regression methods, a diverse range of ML models, including, but not limited to linear models to more complex ensemble model may be used depending on the nature of the ecological, environmental, human migration, animal and insect migration, land use change, and climate change related data and relation with the zoonotic spill-over event. In an embodiment, during the regression and model building, major ecological, environmental, human migration, animal and insect migration, land use change, and/or climate change related change variables may be determined using coefficient estimates and estimated significance, e.g., positive or negative impact to determine the variables having the highest impact. The ML models may include, but not limited to, regression models, such as, Linear Regression, Ridge Regression, Neural Network Regression, Lasso Regression, Elastic Net Regression, Decision Tree Regression, Random Forest, KNN (k-nearest neighbor) Model, Support Vector Machines (SVM), Polynomial Regression, XGboost Regression, Gradient Boosting Regression, CatBoost, and the like; classification models, such as, Logistic Regression, Support Vector Machine, Naive Bayes, Stochastic Gradient Descent Classifier, KNN (k-nearest neighbor), Decision Tree, Random Forest, Gradient Boosting Classifier, XGBoost Classifier, or the like; and anomaly detection models, such as, Angle-Based Outlier Detector (ABOD), K-Nearest Neighbors Detector, Isolation Forest, Histogram-base Outlier Detection (HBOS), Local outlier factor (LOF), DBSCAN, Autoencoders, and the like.

In a non-limiting example, the data analytics function 236 may include query search strings having the ML-based model(s) that are configured to perform at least a bi-variate cluster analysis to identify the ecological, environmental, human migration, animal and insect migration, land use change, and climate change related factors having the highest impact on the historical disease and symptomatology for localit(ies) of interest, e.g., the top five, ten, twenty, thirty, forty, or fifty highest weighted variables. In an embodiment, the bi-variate cluster analysis may be configured to identify features or factors in the data that are as similar as possible, and/or that are different as possible, e.g., effect on the disease incidence and severity. In an embodiment, the data analytics function 236 may include a ML model that is configured to run an inference function in which the endpoint, e.g., historical zoonotic spill-over event based on historical disease incidence and severity, is identified and the ecological, environmental, human migration, animal and insect migration, land use change, and climate change related factors in the data are identified to pass the model, e.g., confidence of prediction of 80%, 85%, 90%, or 95%.

In an embodiment, in order for certain localities of interest to remain anonymous or if real data, e.g., data as discussed above, is not available, synthetic data may be used for the modelling and training of the ML models. Synthetic data is artificial data that is created by using different algorithms that result in data that mirror certain identified statistical properties of the original data. By identifying the important features of population, environmental, ecological, and climate change related data as relevant to a zoonotic spill-over event, the synthetic data may be generated for these relevant features using state-of-art synthetic data generation methods. For example, the synthetic data may be generated, by generating data according to a known distribution, fitting real data to a known distribution, Variational Autoencoder, Generative adversarial network (GAN), or the like. As such, in an embodiment, multiple and separate synthetic data generation methods may be used to produce different datasets of interest (e.g., population, environmental, ecological and climate change related data among others) to avoid the inherent limitations of any one method.

In another non-limiting embodiment, the data analytics function 236 may also include ML model(s) that have been trained and configured to determine a Zoonotic Spill-Over Risk Index, e.g., deployed for inference, for localit(ies) of interest to identify the localities of interest that may have the highest risk for a zoonotic spill-over event, e.g., identifies the top one, two, three, four, five, or six localities, and/or predict the likelihood of a zoonotic spill-over event for a locality of interest. For example, in an embodiment, after the data analytics function 236 has been trained using the bi-variate cluster analysis or iterative correlation and regression analysis functions and weighted to provide a predictive model, a second set of ecological, environmental, human migration, animal and insect migration, land use change, and climate change related data for a second locality of interest may be used to identify other localities of interest that have the highest risk for the zoonotic spill-over event. In an embodiment, the first and second localities of interest may be the same locality of interest. In an another embodiment, the first and second localities of interest may be different localities of interest, e.g., the first locality of interest is used to predictive ML model(s) to determine the Zoonotic Spill-Over Risk Index for another locality of interest.

It is appreciated that the use of a SQL search string with a ML model and/or the use of the Lambda function to call a specific ML model by the data analytics function 236 may depend on a number of various factors. For example, if the data to be analysed is the data from the entire authoritative bucket 220, the Query function may use a SQL string for analysing the data, since SQL strings are more computationally efficient, and may be used for simpler calculations, e.g., initial processing of the data to have the same format. On the other hand, since the ML model(s) called by the data analytics function 236, e.g., a Lambda function, may be used for the bivariate cluster analysis, the data analysed by this ML model(s) may include more specific localities of interest to decrease the data being analysed. As such, the Lambda function may call a plurality of ML model(s) for the bivariate cluster analysis to analyse selected groups of data, e.g., based on localities of interest, to increase computational time and not overburden the computational resources of the cloud-based serverless system.

In a non-limiting embodiment, in view of the size of the data available from the various data source(s) 210, geospatial data may be removed from the data by the data analytics function 236 and/or during the data processing and curation function 225. For example, since during the data processing, the geospatial data may be added as a column to the data tables, by removing the geospatial data from the data, the processing efficiency of the zoonotic spill-over risk analytics and/or prediction process 200 may be improved, at least because the geospatial data would not be included in any of the calculations or ML-based model processing algorithms, e.g., would not slow down processing. In an embodiment, the geospatial data may include spatial data for mapping on a two-dimensional or three-dimensional surface, for example, including, but not limited to, property data including distance, shape, size, relative position, topology of features and boundaries related to mapping. The geospatial data may also include data relating to the specific localit(ies) of interest, which may include identifying information.

In an embodiment, the geospatial data may be reattached after analysis by the data analytics function 236 to provide a mapping and/or visual geospatial representation of the ecological, environmental, human migration, animal and insect migration, land use change, and/or climate change related data and/or Zoonotic Spill-Over Risk Index at different geographical levels for the localities of interest, e.g., via a heatmap. For example, in an embodiment, the Zoonotic Spill-Over Risk Index may be shown as a thematic map showing the risk by area (locality of interest), and can also be presented in tabular and graphical form. The Zoonotic Spill-Over Risk Index may be made relative and normalized to fall into any identified range, in which relative implies that a Zoonotic Spill-Over Risk Index score of “Low” or “12” for a particular area is only meaningful when compared to the risk score of another area that may be “medium” or “17”.

In an embodiment, the resulting analytics data from the data analytics function 236 may be published to a relational database service, e.g., a RDS web-accessible database, and/or a data products web-accessible server, for access by the data consumers via analytics tools 265 for transmitting and/or displaying one or more of the ecological, environmental, human migration, animal and insect migration, land use change, and climate change related factors that have the greatest impact on the prediction of a zoonotic spill-over event, the Zoonotic Spill-Over Risk Index, and associated localities of interest at various viewing levels for displaying on a display with the associated Zoonotic Spill-Over Risk Index, e.g., monitor or display screen. In an embodiment, the data may be accessed by an asynchronous API, webscraping, direct download, and/or operational dashboard, e.g., SAS, Tableau, SQL, Amazon Quicksight or the like, or from cloud objects. In an embodiment, the RDS web-accessible database may be accessible via a server for providing the data to the end user and/or decision makers via a web-accessible server or a REST API. In an embodiment, the RDS web-accessible database may be connected to the server via a host server. As such, the host server may direct traffic or provide accessing credentials for allowing access to the RDS web-accessible server, e.g., username, password, biometrics, encrypted access, or the like.

In an embodiment, the ecological, environmental, human migration, animal and insect migration, land use change, and climate change related factors and their relative impact thereof, the Zoonotic Spill-Over Risk Index, etc., are available for download or accessible through at least one of Esri Marketplace as a Geospatial Layer (or Feature Service), SDI through open standards, including but not limited to WMS, WFS, OGC API, etc., as a CSV or through RESTful and Asynchronous API access, the Esri ArcGIS Server or other geospatial application, or may be viewed as an ArcGIS StoryMap, or an ArcGIS Insights Dashboard, or can be embedded on a public or private (e.g., password-protected) website or web portal. Such access to the data allows the zoonotic spill-over risk analytics and/or prediction process 200 to be configured to adjust the viewability of the ecological, environmental, human migration, animal and insect migration, land use change, and climate change related variables, the Zoonotic Spill-Over Risk Index, etc., from higher levels to granular levels, e.g., from global, national or state level to neighborhood-to-neighborhood, to easily understood the impact of the ecological, environmental, human migration, animal and insect migration, land use change, and climate change related variables on the prediction of a zoonotic spill-over event and/or the localit(ies) having the highest risk of a zoonotic spill-over event occurring so that the decision makers may make actionable decisions to mitigate an effect of the ecological, environmental, human migration, animal and insect migration, land use change, and climate change related variables on the localities of interest. That is, by identifying the impact of the ecological, environmental, human migration, animal and insect migration, land use change, and climate change related variables on the zoonotic spill-over event, a value, e.g., dollar amount, lives saved or other health risk reduction related metric, may be quantified and the return, e.g., economic value, may be determined to assist decision makers in quantifying the effect of the ecological, environmental, human migration, animal and insect migration, land use change, and climate change related variables for improvement thereof.

In an embodiment, the Zoonotic Spill-Over Risk Index may be merged with shape files at multiple geospatial levels (including but not limited to: nation, state, region, county, ZIP code, etc.) for transmission and/or display. The ecological, environmental, human migration, animal and insect migration, land use change, and/or climate change related variables may also be merged with a symbology to illustrate graduation between High risk, Medium risk, and Low risk, in which the symbology can support a wide range of risk levels, typically but not limited to between 3 and 7 levels.

In a non-limiting embodiment, the zoonotic spill-over risk analytics and/or prediction process 200 may be downloadable from the relational database service, e.g., a RDS web-accessible database, and/or the data products web-accessible server and configured as an agent based model. In an embodiment, the zoonotic spill-over risk analytics and/or prediction process 200 may be configured to represent the key components of the system, such as animals, humans, pathogens, and the environment, e.g., via interactive GUI. Each component may be represented as an agent with specific attributes, such as location, behavior, and health status. The model may be parameterized or weighted based on the data and information on the system, e.g., pre-trained, such as the distribution of animal and human populations, the transmission pathways of pathogens, the environmental conditions, and the disease incidence and severity. The model may be deployed for inference to simulate the spread of zoonotic diseases from animals to humans over time. The model may use ML algorithms to simulate the behavior and interactions of the agents, as well as the transmission and progression of the disease, as in the various models and algorithms discussed above. The key components of the system may be changed, e.g., added/removed, via the interactive GUIs to determine the effect, e.g., direction and magnitude, of the various agents on the prediction of the zoonotic spill-over event, e.g., the spread of the zoonotic disease.

In another non-limiting embodiment, since data from the data source(s) 210 may be obtained and accessed as streaming data, the data analytics function 236 may be configured to perform additional processing from the data relational database service and/or a data products web-accessible server. For example, in an embodiment, the zoonotic spill-over risk analytics process 200 may be configured to track the impact of the interventions or mitigation effects in the short- and long-term. This allows for immediate feedback to course correct interventions, helping ensure successful mitigation of the identified ecological, environmental, human migration, animal and insect migration, land use change, and/or climate change related factors that affect zoonotic spill-over events or prediction thereof. Such tracking may be based on ML models that are configured to predict the effect of the mitigation of the one or more ecological, environmental, human migration, animal and insect migration, land use change, and/or climate change related factors at the locality of interest based on prior effects on the zoonotic spill-over event. Such predictions by the ML models of the data analytics function 236 may be used to provide a triggering event determination from the ecological, environmental, human migration, animal and insect migration, land use change, and/or climate change related factors that either changes the view of the zoonotic spill-over risk analytics and/or prediction process 200, e.g., expanded or contracted viewing, to provide the decision maker with an actionable view and value in an attempt to further mitigate the ecological, environmental, human migration, animal and insect migration, land use change, and/or climate change related factors that affect zoonotic spill-over events. The triggering event may be based on threshold values based on the distribution of the risk signal. For example, in an embodiment, the Zoonotic Spill-Over Risk Index values below 2.5 standard deviations may be categorized as low risk, whereas, the values above 2.5 standard deviations may be categorized as high risk, which would result in a triggering event. As such, in an embodiment, if the Zoonotic Spill-Over Risk Indexes are trending in a positive direction, the data analytics function 236 may be configured to predict the Zoonotic Spill-Over Risk Index as a triggering event to inform the decision maker to take an appropriate action. In yet another embodiment, since the zoonotic spill-over risk analytics and/or prediction process 200 is receiving streaming data, when the localities of interest change having the highest zoonotic spill-over risk index changes, e.g., due to changes in the ecological, environmental, human migration, animal and insect migration, land use change, and climate change related variables at a locality of interest, any updated localities of interest having a new highest zoonotic spill-over risk index and new ecological, environmental, human migration, animal and insect migration, land use change, and climate change related variables associated with the highest impact associated with the new highest zoonotic spill-over risk index may be transmitted or displayed, e.g., in the data relational database service and/or data products web-accessible server and/or display for the decision maker.

In another non-limiting embodiment, the zoonotic spill-over risk analytics and/or prediction process 200 may be downloadable and include the ML model as a predictive model that may include a plurality of analyser channels (customized for the zoonosis), each of which corresponds to an observable condition of a patient, e.g., symptomology, and/or ecological, environmental, human migration, animal and insect migration, land use change, and climate change related data relevant to the locality of interest. The channels may be weighted to customize or fine tune the predictive model, signifying whether any channels are of equal or greater/lesser importance than others in identifying the prediction of the zoonotic spill-over event.

Predictive modelling may allow allocation of channel points in accordance with, or independent of, channel weighting based on the statistical sensitivity of specific factors in predicting. For example, a base score may be calculated as the summation of points attributed to the (weighted or unweighted) analyser channels. The analyser channels may be broken down further into analyser features that provide additional sensitivity in identifying localities of interests that may be more prone to be affected by the zoonosis.

In one or more embodiments, points and/or weights may be assigned to each channel. It should be noted that not all of the channels or features need be part of any given analysis. Moreover, other channels and/or features may be suitable in addition or in the alternative, depending on the study or analysis. In one or more embodiments, point modifiers may be applied to one or more of the channels and/or features to affect the influence of the same on the total base score. Non-limiting examples include percentage weightings, inclusion/exclusion of certain channels/features to suit any particular analysis or subject population, etc.

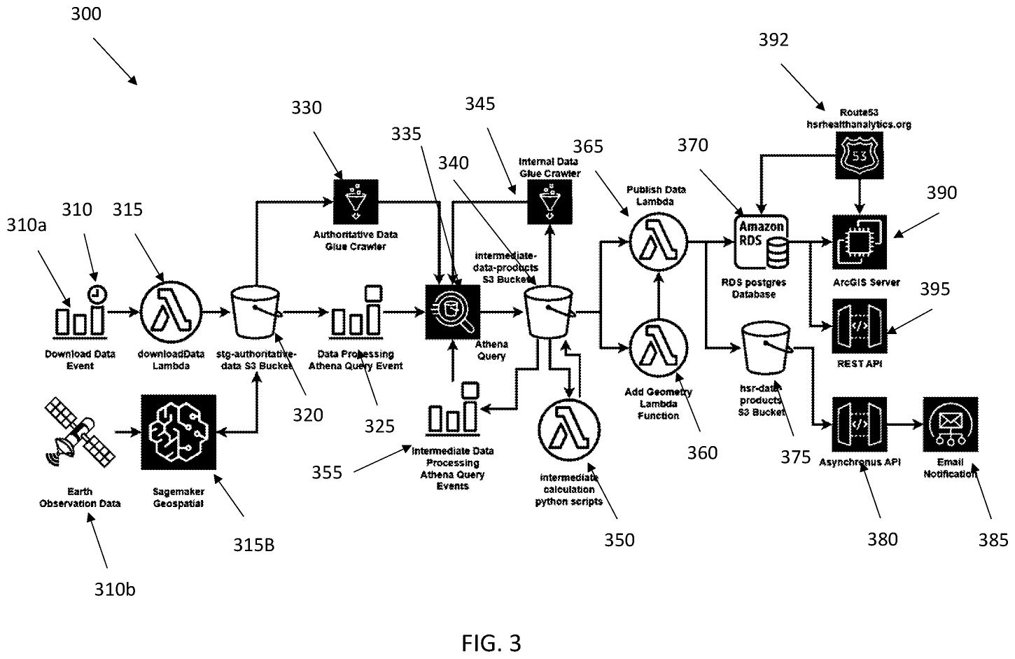

FIG. 3 is an illustration of a zoonotic spill-over risk analytics and/or prediction system 300, according to at least another embodiment described herein. The system 300 may be implemented by a program, custom circuits, or by a combination thereof. The system 300 may include data producers 310, a data pipeline that includes the data processing and analytics, e.g., 315, 335, 340, 345, 350, 355, and data storage, e.g., 320, 370, 375, and data consumers, e.g., 390, 395, 380, 385, for accessing the resulting determinations. The system 300 may be configured to determine which ecological, environmental, human migration, animal and insect migration, land use change, and climate change related factors, or other factors, impact the prediction of zoonotic spill-over events and/or predictive risk of a locality of interest of a zoonotic spill-over event, in which the resulting risk determination may be adjustable, e.g., be provided with expanded or contracted viewing, for viewing at national, global, or regional levels to viewing at various localities. The system 300 may be a serverless, e.g., cloud-based, query service system, a cloud-based risk analysis framework system, a data lake infrastructure that allows for the curation, cleaning, analysis, and dissemination of zoonotic spill-over risk analytics and/or prediction, a cloud-based spatial data infrastructure, or a cloud-based system with API-enabled accessibility, or a combination of thereof. The system 300 may have an infrastructure that is uniquely designed by including a purpose-built extract, transform and load (ETL) dataflow pipeline that may include, but not limited to, S3, Athena Query, Glue, Lambda, RDS, SageMaker, SageMaker Geospatial, among others, in which the dataflow pipeline may be built on script using Python, SQL, and utilize various packages including numpy, pandas, geopandas, matplotlib, seaborn, etc. The system may leverage a bi-variate cluster analysis that includes Al-based machine learning models to identify the specific ecological, environmental, human migration, animal and insect migration, land use change, and climate change related factors or other factors that impact the risk of zoonotic spill-over events and/or may be used to predict the risk of a locality of interest of a zoonotic spill-over event.

FIG. 3 shows a plurality of data sources 310a, 310b, . . . 310n (collectively, “data sources 310” hereafter), which may be communicatively coupled to the zoonotic spill-over risk analytics and/or prediction system 300. Data sources 310a, 310b, . . . 310n may be the same as databases 210 of FIG. 2 discussed above and may include to, but not be limited to, e.g., Census data, foot traffic data/mobility data, human population and growth data, earth observation data from satellite imagery, including, but not limited to cloud cover, weather patterns, deforestation, vegetation, multispectral imaging, natural disasters, such as, volcanic eruptions, wildfires, and flooding, effects of climate change, or the like, global mammalian richness data, human mobility data, environmental data, genomic surveillance data, sampling data from animal populations, ground-level data, e.g., from road networks, Google Maps, or Open Street Maps, emerging and re-emerging infectious disease data, deforestation data, proximity of humans to wildlife data, rainfall, temperature, or other weather related dated, historical data on infection/disease incidence, historical data on symptomatology, etc. Further, not only are the systems described, recited, and foreseen herein not limited to the data sources listed above, but they are not limited in quantity to those shown in FIG. 3. Further still, unless context otherwise requires, the description and recitation henceforth may refer to the singular “data source 310” without being limiting.

The various data sources 310 may be accessed and obtained by the data pipeline of the zoonotic spill-over risk analytics and/or prediction system 300 by querying the data sources 310, for example, by using a fetcher function having a Lambda function 315, e.g., Python script, having for example, a SQL search string to obtain a set of the ecological, environmental, human migration, animal and insect migration, land use change, and climate change related data for the localities of interest, e.g., national, regional, state, neighborhood-by-neighborhood, or a combination thereof. For example, the Lambda function 315 may be configured to call a function and/or algorithm to direct the obtainment of data from any number of the available data sources 310 around the world by focusing on the localities of interest that are more at risk than others, e.g., where risk is high or may be high. In an embodiment, the Lambda function 315 may include an ingestion function that includes Amazon SageMaker Geospatial to process the earth observation data. In another embodiment, the data may be selectively obtained over the worldwide web and accessed using APIs, web scraping functionalities, direct download of files, and other cloud-based objects. The data may be stored in an authoritative bucket 320, as raw data, e.g., data from the data sources without any further processing.

The data pipeline of the zoonotic spill-over risk analytics and/or prediction system 300 may further include a data processing query event 325 that is configured to scrub data, e.g., metadata, from the data in the authoritative bucket 320. As such, the data from the data sources 310 does not include metadata, e.g., source identifying data, such that the data that is processed in the zoonotic spill-over risk analytics and/or prediction system 300 is secured and the data source may remain anonymous, e.g., source identifying information is removed. In an embodiment, the data processing query event 325 may include an algorithm that is configured to convert the data into a risk signal, in which the underlying data is erased for further security and anonymity of the data and source data. For example, the data processing query event 325 may invoke a signal extraction function, e.g., trending of the data in a positive or negative direction, such that the risk of the factor may still be assessed, but the context of the data is removed, e.g., to provide security of the data, for example, specific country to be assessed. In an embodiment, a crawler function 330 may be used that is configured to obtain the data from the authoritative bucket 320 to populate the data for analysis in the zoonotic spill-over risk analytics and/or prediction system 300. The crawler function 330 may be configured to convert the data into tabular form in which any geospatial data is removed. The crawler function 330 may be configured to crawl the data sources 310 in a single run such that the crawler function 330 may determine the format, schema, and associated properties in the raw data in the authoritative bucket 320, group the data into tables or partitions, and write or add data for analysis. It is appreciated that the geospatial data may include data about objects, events, or phenomena that have a location on the surface of the earth, e.g., a geographic component, that may include geometric data, e.g., data as 2D and/or 3D vectors as points, lines, and polygons in a space, cartographic representations, and/or other positional relationship data.

The data processing query event 325 and/or the crawler function 330 may then be configured to populate the respective transformed data to a Query function 335 having memory to store the data, for example, in a hive data format, e.g., suitable format for processing in intermediate data bucket 340.

In a non-limiting example, the Query function 335 may be connected in a loop with the intermediate data bucket 340, an internal crawler function 345, an intermediate calculation function 350, and an intermediate data processing event function 355, in which a number of different processing steps may occur on the intermediate data. The Query loop may include a plurality of artificial intelligence based Machine-Learning Models that may be included in search functions, e.g., SQL search strings, that is configured to access the hive data and perform at least the following functions/processing. In an embodiment, the Query loop may be configured to remove erroneous data, ensure the data has proper geocoding data and format, make necessary corrections to an imagery data obtained, e.g., atmospheric data correction of satellite imagery, for example, remove cloud cover, process any imagery data, e.g., using SageMaker Geospatial, ENVI, or ARTS software, stitch any imagery data together, label and identify the data, e.g., tagged data from the web or identify the data, e.g., as a wildfire or urban encroachment. In an embodiment, the Query loop may be configured to convert the imagery data and/or combine imagery data together and covert the image data into tabular form, e.g., using SageMaker Geospatial, or ENVI or ARTS software. As such, the image data may be processed into numerical form using the Query function 335 such that the image data may be considered in determining the effect of the ecological, environmental, human migration, animal and insect migration, land use change, or climate change related factors presented in the image data on the prediction or occurrence of the zoonotic spill-over event. For example, in an embodiment, the image data may include images of deforestation, wildfire, vegetation, or urban encroachment for a locality of interest. As such, such data may be converted into tabular form and then analysed using the Al-enabled ML algorithm to determine the effects thereof.

The Query function 335 may include query search strings having ML-based models that are configured to perform analysis at the global level, e.g., on the hive data, in the intermediate data bucket 340. In an embodiment, the query search string may be a SQL search that may be used to identify the ecological, environmental, human migration, animal and insect migration, land use change, or climate change related factors having the greatest impact on the zoonotic spill-over event at a global or high level of the data in the intermediate data bucket 340.

In an embodiment, the internal crawler function 345 may be configured to obtain the data from the intermediate data bucket 340 and may be configured to convert the data into a form for data analysis and back into tabular form. The crawler function 345 may be configured to determine the format, schema, and associated properties in the intermediate data in the intermediate data bucket 340, group the data into tables or partitions, and write or add data for analysis.

The intermediate calculation function 350 may include a plurality of functions/algorithms, including, but not limited to, a ML/AI algorithm or other data science technique for bi-variate cluster analysis that are configured to identify the ecological, environmental, human migration, animal and insect migration, land use change, or climate change related factors that have the highest impact on the zoonotic spill-over event for the particular locality of interest, e.g., the top five, ten, twenty, thirty, forty, or fifty highest weighted variables. The bi-variate cluster analysis may be configured to identify features or factors in the data that are as similar as possible, and/or that are different as possible. The bi-variate cluster analysis may also include a varying coefficient model, weighted distance measuring model, K-means and hierarchical clustering models, or the like for processing the data. For example, a series of the ML/AI algorithms having the bivariate cluster analysis may be run, for example, an inference function in which the endpoint, e.g., zoonotic spill-over event, is identified and the ecological, environmental, human migration, animal and insect migration, land use change, or climate change related factors in the data are identified to pass the model, e.g., using Amazon SageMaker and/or Amazon SageMaker Geospatial to deploy various functions. In an embodiment, the data may be gathered and analysed for the different levels of the localities of interest such that the data is available or accessible for the different levels of the localities of interest.

In an embodiment, the intermediate calculation function 350 may also include ML model(s) that have been trained and is configured to determine a Zoonotic Spill-Over Risk Index, e.g., deployed for inference, for the localit(ies) of interest to identify the localities of interest that may have the highest risk for a zoonotic spill-over event, e.g., identifies the top one, two, three, four, five, or six localities, and/or predict the likelihood of a zoonotic spill-over event for a locality of interest. For example, in an embodiment, after the intermediate calculation function 350 has been trained using the bi-variate cluster analysis or iterative correlation and regression analysis functions and weighted to provide a predictive model, a second set of ecological, environmental, human migration, animal and insect migration, land use change, and climate change related data for a second locality of interest may be used to identify other localities of interest that have the highest risk for the zoonotic spill-over event. In an embodiment, the first and second localities of interest may be the same locality of interest. In an another embodiment, the first and second localities of interest may be different localities of interest, e.g., the first locality of interest is used to predictive ML model(s) to determine the Zoonotic Spill-Over Risk Index for another locality of interest.

In an embodiment, in view of the size of the data available from the various data sources 310, geospatial data may be removed from the data using at least one of the Query function 335, e.g., via SQL string, and the intermediate data processing event function 355. For example, since during the data processing the geospatial data may be added as a column to the data tables, by removing the geospatial data from the data, the processing efficiency of the zoonotic spill-over risk analytics and/or prediction system 300 may be improved, at least because the geospatial data would not have been included in any of the calculations or ML/Al-based model processing algorithms. In an embodiment, the geospatial data may include spatial data for mapping on a two-dimensional or three-dimensional surface, for example, including, but not limited to, property data including distance, shape, size, relative position, topology of features and boundaries related to mapping. The geospatial data may also include data relating to the specific localit(ies) of interest.

In an embodiment, the intermediate calculation function 355 may also be configured to remove erroneous data, ensure the data has proper geocoding data and format, make necessary corrections to an imagery data obtained, e.g., atmospheric data correction of satellite imagery, for example, remove cloud cover, process any imagery data, e.g., using SageMaker Geospatial, or ENVI or ARTS software, stitch any imagery data together, label and identify the data, e.g., tagged data from the web or identify the data, e.g., as a wildfire or deforestation.

In an embodiment, the intermediate data bucket 340 may be accessed by an add geometry function 360 which is configured to convert the data in tabular form from the intermediate data bucket 340. The add geometry function 360 may also be configured to add any geospatial data that was previously removed by the Query function 335 and/or the intermediate data processing event function 355. A publish data function 365 may be used to publish the data processed by the query loop, e.g., the identification of the ecological, environmental, human migration, animal and insect migration, land use change, or climate change related factors that have the greatest impact on the prediction of the zoonotic spill-over event and their relative impact thereof, the Zoonotic Spill-Over Risk Index, and associated localities of interest at various viewing levels for displaying on a display with the associated Zoonotic Spill-Over Risk Index, e.g., monitor or display screen of the decision maker. The publish data function 365 may be configured to send or receive a signal to send the ecological, environmental, human migration, animal and insect migration, land use change, or climate change related factors, the Zoonotic Spill-Over Risk Index, etc., to a relational database service, e.g., RDS web-accessible database 370, and/or a data products web-accessible server 375 that includes databases in the cloud such that the data may be accessed by an end user and/or a public health decision maker for displaying on a display. In an embodiment, the data may be accessed by an asynchronous API 380 that is connected to an email server 385 to send the data or link to the data to the end user or the public health decision maker. In an embodiment, the RDS web-accessible database 370 may be accessible via a server 390 for providing the data to the end user and/or public health decision maker via a web-accessible server or a REST API 395. In an embodiment, the RDS web-accessible database 370 may be connected to the server 390 via a host server 392. As such, the host server 392 may direct traffic or provide accessing credentials for allowing access to the RDS web-accessible server 370, e.g., username, password, biometrics, encrypted access, or the like.

In an embodiment, the ecological, environmental, human migration, animal and insect migration, land use change, or climate-change related factors and their relative impact thereof, the Zoonotic Spill-Over Risk Index, etc., are available for download or accessible through at least one of Esri Marketplace as a Geospatial Layer (or Feature Service), SDI through open standards, including but not limited to WMS, WFS, OGC API, etc., as a CSV or through RESTful and Asynchronous API access, the Esri ArcGIS Server or other geospatial application, or may be viewed as an ArcGIS StoryMap, or an ArcGIS Insights Dashboard, or can be embedded on a public or private (e.g., password-protected) website or web portal. Such access to the data allows the zoonotic spill-over risk analytics and/or prediction system 300 to be configured to adjust the viewability of the ecological, environmental, human migration, animal and insect migration, land use change, and climate change related variables, the Zoonotic Spill-Over Risk Index, etc., from higher levels to granular levels, e.g., from global, national or state level to neighborhood-to-neighborhood, to easily understood the impact of the ecological, environmental, human migration, animal and insect migration, land use change, and climate change related variables on the prediction of a zoonotic spill-over event and/or the localit(ies) having the highest risk of a zoonotic spill-over event occurring so that the decision makers may make actionable decisions to mitigate an effect of the ecological, environmental, human migration, animal and insect migration, land use change, and climate change related variables on the localities of interest. That is, by identifying the impact of the ecological, environmental, human migration, animal and insect migration, land use change, and climate change related variables on the zoonotic spill-over event, a value, e.g., dollar amount, lives saved or other health risk reduction related metric, may be quantified and the return, e.g., economic value, may be determined to assist decision makers in quantifying the effect of the ecological, environmental, human migration, animal and insect migration, land use change, and climate change related variables for improvement thereof.

In an embodiment, the Zoonotic Spill-Over Risk Index may be merged with shape files at multiple geospatial levels (including but not limited to: nation, state, region, county, ZIP code, etc.) for transmission and/or display. The ecological, environmental, human migration, animal and insect migration, land use change, and/or climate change related variables may also be merged with a symbology to illustrate graduation between High risk, Medium risk, and Low risk, in which the symbology can support a wide range of risk levels, typically but not limited to between 3 and 7 levels.

In a non-limiting embodiment, the zoonotic spill-over risk analytics and/or prediction process 300 may be downloadable and configured as an agent based model. In an embodiment, the zoonotic spill-over risk analytics and/or prediction process 300 may be configured to represent the key components of the system, such as animals, humans, pathogens, and the environment, e.g., via interactive GUI. Each component may be represented as an agent with specific attributes, such as location, behavior, and health status. The model may then be parameterized or weighted based on the data and information on the system, such as the distribution of animal and human populations, the transmission pathways of pathogens, the environmental conditions, and the disease incidence and severity. The model may be deployed for inference to simulate the spread of zoonotic diseases from animals to humans over time. The model may use ML algorithms to simulate the behavior and interactions of the agents, as well as the transmission and progression of the disease, as in the various models and algorithms discussed above. The key components of the system may be changed, e.g., added/removed, via the interactive GUIs to determine the effect, e.g., direction and magnitude, of the various agents on the prediction of the zoonotic spill-over event, e.g., the spread of the zoonotic disease.

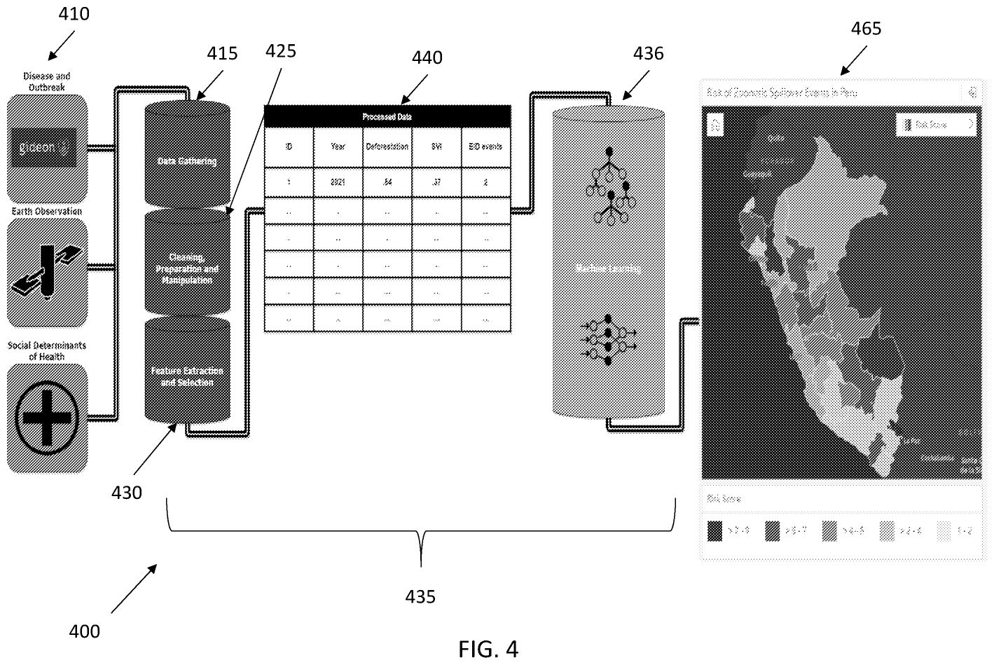

FIG. 4 is an example embodiment of a zoonotic spill-over risk analytics and/or prediction system 400, which may have the same or similar features as described above with respect to systems 200 and 300. In this embodiment, this risk mapping may be paired with a public health model using historical disease and symptomatology data from electronic health records systems and other sources to detect clusters of new symptoms among patients for the purpose of anomaly detection to inform public health decision makers and appropriate parties on where potential outbreaks may be occurring. The system 400 may be implemented by a program, custom circuits, or by a combination thereof. The system 400 may be a serverless, e.g., cloud-based, query service system, a cloud-based risk analysis framework system, a data lake infrastructure that allows for the curation, cleaning, analysis, and dissemination of zoonotic spill-over risk analytics and/or prediction, a cloud-based spatial data infrastructure, or a cloud-based system with API-enabled accessibility, or a combination of thereof. The system 400 may include data producers 410, a data pipeline that includes the data processing and analytics, e.g., 415, 425, 430, 435, 440, 436, and data consumers, e.g., 465, for accessing the resulting determinations.

FIG. 4 shows a plurality of data sources 410. Data sources 410 may be the same as databases 210 of FIG. 2 or 310 of FIG. 3 discussed above and may refer to, but not be limited to, disease and outbreak data, e.g., from GIDEON, earth observation, e.g., satellite imagery from Sentinel and/or LandSAT, electronic health records, and social determinants of health, e.g., social and economic factors that affect health outcomes, as described in U.S. application Ser. No. 18/061,686, filed Dec. 5, 2022, which is incorporated by reference. In an embodiment, the data sources 410 may include data of human population and growth, mobility data, global mammalian richness, emerging infectious disease events, deforestation, proximity of humans to wildlife, rainfall, historical data on infections/disease incidence, and historical data on symptomology. To the infections/disease incidence and historical data on disease symptomology can be applied clustering and classification algorithms to detect and understand where potentially anomalous clusters of symptoms are occurring. Layered on top of the risk maps will result in a more clear understanding if a potential zoonotic spill-over event has occurred. Further, not only are the systems described, recited, and foreseen herein not limited to the data sources listed above, but they are not limited in quantity to those shown in FIG. 4. Further still, unless context otherwise requires, the description and recitation henceforth may refer to the singular “data source 410” without being limiting.

The various data source(s) 410 may be accessed and obtained by the processing system 435 by querying the data source(s) 410, for example, by including and using a data gathering function 415. The data gathering function 415 may include a Lambda function, e.g., Python script, having a SQL search string to access and obtain a set of ecological, environmental, human migration, animal and insect migration, land use change, climate change related, and health data, such as claims data or electronic health records data, for the localities of interest from the data source(s) 410. In an embodiment, the data gathering function 415 may additionally include an extract, transform, and/or load service, e.g., Apache Kafka, Amazon Kinesis Firehouse, SageMaker Geospatial, or the like, to obtain and access the data source(s) 410 as streaming data and/or for enhancing or converting the geospatial data into numeric or tabular form.

The processing system 435 may further include a data cleaning, preparation, and manipulation function 425 that may be configured to scrub data, e.g., metadata, from the data, after the data is gather by the data gathering function 415, e.g., stored data. As such, the data from the data source(s) 410 may not include metadata, e.g., source identifying data, such that the data that is processed in the processing system 435 is secured and the data source may remain anonymous, e.g., source identifying information is removed. In an embodiment, the data cleaning, preparation, and manipulation function 425 may be configured to convert the data into a risk signal, in which the underlying data is erased. For example, the data cleaning, preparation, and manipulation function 425 may invoke an algorithm that is configured as a signal extraction function, e.g., trending of the data in a positive or negative direction, such that the risk of the factor may still be assessed, e.g., magnitude in the positive or negative direction, but the context of the data is removed, e.g., to provide security of the data. The data cleaning, preparation, and manipulation function 425 may include algorithms written in object-oriented programming and functional programming scripts, e.g., Scala, Python, Node (Java), and/or accessed by open-source, distributed processing system, such as, Apache Spark, etc., for development of APIs in Java, Python, Scala, R, or the like.

In an embodiment, a feature extraction and selection function 430, e.g., a crawler function, may be used that is configured to obtain the data after the data is cleaned, prepared, and manipulated by the data cleaning, preparation, and manipulation function 425 to populate the data for analysis in the processing system 435. The feature extraction and selection function 430 may be configured to convert the data into tabular form, e.g., table 440, in which any geospatial identification data may be removed and the data is converted to numerical form. The feature extraction and selection function 430 may be configured to crawl the data source(s) 410 in a single run such that the feature extraction and selection function 430 may determine the format, schema, and associated properties in the raw data, group the data into tables or partitions, and write or add data for analysis. In an embodiment, the feature extraction and selection function 430 may include AWS SageMaker Geospatial or other image processing platform to access, interact, and interrogate imagery, including, but not limited to satellite imagery, and convert the imagery to numerical/tabular form, in addition to algorithms for converting the data into comparative formats.

The data cleaning, preparation, and manipulation function 425 and/or the feature extraction and selection function 430 may then be configured to populate the respective transformed data for analysis by a data analytics function 436 having memory to store the data, for example, in a hive data format.

The data analytics function 436 may be used to generate a Zoonotic Spill-Over Risk Index which may be an indication of which ecological, environmental, human migration, animal and insect migration, land use change, and climate change related variables have the highest impact on a zoonotic spill-over event and/or a prediction of an occurrence of a zoonotic spill-over event. In an embodiment, the data analytics function 436 may include a machine learning (ML) model in which the training data or the units of observation for the model may be historical data on grid cells representing land areas where there have been emerging infectious diseases. The features are the grid cell's corresponding ecological and climate change related variables. The ML model may include a regression classification algorithm to determine the relative weights for each of the variables or features included in the model. These weights may then be used to create a heatmap of the relative risk of a zoonotic spill-over, as discussed further below.

In a non-limiting example, the data analytics function 436 may be a loop that includes a plurality of artificial intelligence based ML Models that are included in search functions, e.g., SQL search strings and/or Lambda functions for calling the ML models that are connected in nodes, that is configured to access the hive data and perform at least the following functions/processing. The ML models may include, but not limited to, regression models, such as, Linear Regression, Ridge Regression, Neural Network Regression, Lasso Regression, Elastic Net Regression, Decision Tree Regression, Random Forest, KNN (k-nearest neighbor) Model, Support Vector Machines (SVM), Polynomial Regression, XGboost Regression, Gradient Boosting Regression, CatBoost, and the like; classification models, such as, Logistic Regression, Support Vector Machine, Naive Bayes, Stochastic Gradient Descent Classifier, KNN (k-nearest neighbor), Decision Tree, Random Forest, Gradient Boosting Classifier, XGBoost Classifier, or the like; and anomaly detection models, such as, Angle-Based Outlier Detector (ABOD), K-Nearest Neighbors Detector, Isolation Forest, Histogram-base Outlier Detection (HBOS), Local outlier factor (LOF), DBSCAN, Autoencoders, and the like.

The data analytics function 436 may also include query search strings having ML-based models that are configured to perform at least a bi-variate cluster analysis to identify the ecological, environmental, human migration, animal and insect migration, land use change, and climate change related factors having the highest impact on the prediction or occurrence of a zoonotic spill-over event. In an embodiment, the bi-variate cluster analysis may be configured to identify features or factors in the data that are as similar as possible, and/or that are different as possible, e.g., effect on the zoonotic spill-over event or prediction thereof, including, but not limited to disease incidence and severity, at various levels of the localities of interest, e.g., global, national, state, or regional and ZIP Code, from epidemiology reports. In an embodiment, the top five, ten, twenty, thirty, forty, or fifty highest weighted variables may be used by the ML model(s). The bi-variate cluster analysis may also include a varying coefficient model, weighted distance measuring model, K-means and hierarchical clustering models, or the like for processing the data.

The data analytics function 436 may include a ML/AI or data science algorithm that is configured to run an inference function in which the endpoint, e.g., zoonotic spill-over event, is identified and the ecological, environmental, human migration, animal and insect migration, land use change, and climate change related factors in the data are identified to pass the model, e.g., training using Amazon SageMaker and/or Amazon SageMaker Geospatial to deploy the various algorithms/functions. The ML/Al algorithm may also include the SQL search strings in which the ML/AI algorithm is configured to determine which of the ecological, environmental, human migration, animal and insect migration, land use change, and climate change related variables have the highest impact on the zoonotic spill-over event or prediction thereof. In an embodiment, the data is gathered and analysed for the different levels of the localities of interest such that the data is available or accessible for the different levels of the localities of interest.

In an embodiment, data analytics function 436 may include one or more of the following variables: ecological, environmental, human migration, animal and insect migration, land use change, and climate change related factors determined from the Journal of Infectious Disease (controlling for spatial and temporal bias), human population growth, emerging and infectious disease events, indicator(s) of rapid human population growth, wildlife host species richness, mobility data, deforestation data, proximity of humans to wildlife, sampling/testing data, e.g., from DEEP VZN, average rainfall, and the like. The data analytics function 436 may then use a ML model that includes a logistic regression of the variables, such as the following:

$$log ( pi / 1 - pi ) ( EID ) = B 0 + B ( JID ) + B ( Population Growth ) + B ( EID ) + B ( Indicator Population Growth ) + B ( Host Species Richness ) + B ( Mob Data ) + B ( Deforst ) + B ( Sample / Test ) + B ( Prox to Wildlife ) + B ( Avg Rainfall )$$

The logistic regression may also include ML models that further include historical rates of infection and symptomology for a plurality of diseases.

As such, based in the ecological, environmental, human migration, animal and insect migration, land use change, climate change related data, and historical diseases, the ecological, environmental, human migration, animal and insect migration, land use change, and climate change related variables having the highest impact on the indication and/or prediction of the zoonotic spill-over event may be identified and quantified, e.g., the top five, ten, twenty, thirty, forty, or fifty highest weighted variables. Moreover, the data analytics function 436 may include trained ML models that may be deployed for inference that predict future zoonotic spill-over events, e.g., future infection incidences, for a second set of data or streaming data.

In an embodiment, the resulting analytics data from the data analytics function 436 may be published to a relational database service, e.g., a RDS web-accessible database, and/or a data products web-accessible server, for access by the data consumers via analytics tools 465 for transmitting and/or displaying one or more of the ecological, environmental, human migration, animal and insect migration, land use change, and climate change related factors that have the highest impact on the prediction of a zoonotic spill-over event, the Zoonotic Spill-Over Risk Index, and associated localities of interest at various viewing levels for displaying on a display with the associated Zoonotic Spill-Over Risk Index, e.g., monitor or display screen. In an embodiment, the data may be accessed by an asynchronous API and/or operational dashboard, e.g., SAS, Tableau, SQL, Amazon Quicksight or the like.

That is, after the ML models have been trained in the data analytics function 436, the ML models may be used with patient level data to predict disease incidence based on the historical symptomatology and disease data to understand where anomalous clusters of symptoms are occurring. This may then be layered on top of the existing risk maps to have a clearer understanding if a zoonotic spill-over event has occurred and/or whether a zoonotic spill-over event may occur for a specific locality of interest, e.g., Zoonotic Spill-Over Risk Index. The Zoonotic Spill-Over Risk Index may then be used to designate where early surveillance interventions could be used and/or should be directed. For example, in an embodiment, To the infections/disease incidence and historical data on disease symptomology can be applied clustering and classification algorithms to detect and understand where potentially anomalous clusters of symptoms are occurring. Layered on top of the risk maps will result in a more clear understanding if a potential zoonotic spill-over event has occurred.

In a non-limiting embodiment, the analytics tool 465 may be a GUI in the form of a heatmap to display the varying levels of risk for a locality of interest, e.g., Peru. The GUI may be interactive and configured to allow selection specific areas in the locality of interest to investigate the ecological, environmental, human migration, animal and insect migration, land use change, and climate change related data particularly relevant, e.g., highest impact thereon, in the specific area, and to identify and understand the factors underlying the identified Zoonotic Spill-Over Risk index, e.g., the risk level. In another non-limiting embodiment, the GUI may also include a time and a date range parameter to show how the ecological, environmental, human migration, animal and insect migration, land use change, and climate change related data, e.g., deforestation, land use change, melting permafrost, etc., in a particular region lead to the zoonotic spill-over event. In an embodiment, when the data is processed continuously, e.g., streaming, the localities of interest and/or specific areas may be automatically identified, e.g., size of the area is increased, or automatically categorized with the areas having the greatest Zoonotic Spill-Over Risk Index.

As such, the analytics tool 465 may be configured to quickly visually identify the causative factors/variables that may be instructive in designing a mitigation that may be effective to prevent or mitigate the zoonotic spill-over event. In addition, the analytics tool 465 may be configured to inform the degree to which each causative factor/variable is contributing to the overall zoonotic spill-over risk, which may inform the potential risk reduction that can be accomplished by mitigating that factor.

While the above factors have been discussed, it is understood that the factors are not an exhaustive list of the factors that may be considered. In an embodiment, other factors may be considered that are more important and/or more instructive on the zoonotic spill-over event. In an embodiment, the zoonotic spill-over risk analytics and/or prediction process or system may include multiple output layers that may be selected from a drop down menu in a base dashboard. The zoonotic spill-over risk analytics and/or prediction process or system may be configured to import or add additional data, e.g., users can bring their own data to overlay on top of any of the output products. The zoonotic spill-over risk analytics and/or prediction process or system may also be configured to filter the display of the data by administrative level and through a designated area of interest which can be a circle, square, hand-drawn shape, or polygon to select the data sources for training and/or analysis by the zoonotic spill-over risk analytics and/or prediction process or system.

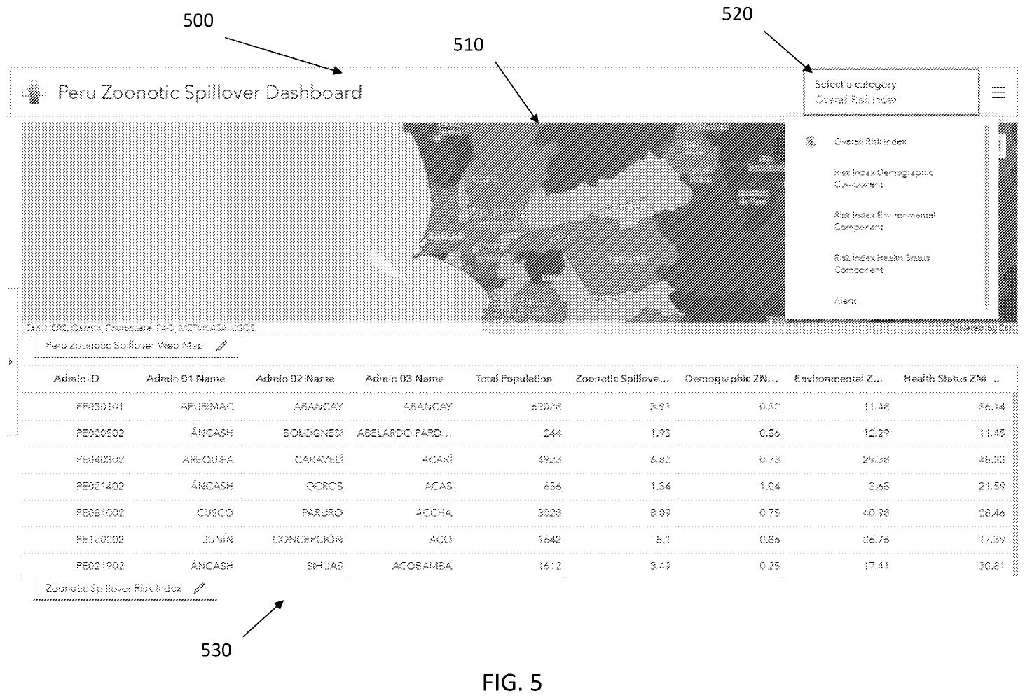

FIGS. 5, 6, and 7 are illustrated views of a zoonotic spillover dashboard 500 in which a user can select to view the overall index or data specific to an individual category of risk for transmitting and/or displaying one or more of the ecological, environmental, human migration, animal and insect migration, land use change, and climate change related factors that have the highest impact on the prediction of a zoonotic spill-over event, the Zoonotic Spill-Over Risk Index, and associated localities of interest at various viewing levels for displaying on a display with the associated Zoonotic Spill-Over Risk Index, e.g., monitor or display screen. In an embodiment, the dashboard 500 for the Zoonotic Spill-Over Risk Index may include a thematic map viewing area 510, a menu selection 520 for interactive viewing of displayed information for the dashboard 500, and information display area 530.

In an embodiment, the thematic map viewing area 510 may include the risk of zoonotic spill-over for the specified geography, e.g., local to global, e.g., for the nation of Peru, in which different colors may be provided to provide visual indication of risk areas for localities. In some embodiments, depending the interactive menu, the dashboard 500 may show multiple pieces of information simultaneously, based on user selection of specific information from the menu 520, e.g., Overall Risk Index, Risk Index Demographic Component, Risk Index Environmental Component, Risk Index Health Status Component, and Alerts, such that different data and information may be shown, and in different formats, to help guide the analysis into the zoonotic spill-over risk itself, e.g., the contributing factors—as well as into understanding the key factors driving the risk, e.g., the ecological, environmental, human migration, animal and insect migration, land use change, and climate change related factors that have the highest impact on the prediction of a zoonotic spill-over event and/or the Zoonotic Spill-Over Risk Index.

For example, in an embodiment, as illustrated in FIG. 5, by selecting the “Overall Risk Index” option on the “Select a Category” drop down menu 520 (upper right corner)—the dashboard shows the thematic map 510 for Peru along with a Zoonotic Spill-Over Risk Index table in the information display area 530 that identifies the values of each component of the risk by each Administrative III Unit (i.e., the equivalent of Census tracts in the U.S.) within Peru. In some embodiments, the available options in this pull-down menu are configurable, e.g., user defined and/or determined for a specific user or locality of interest, e.g., different countries or regions may require different identification of factors.

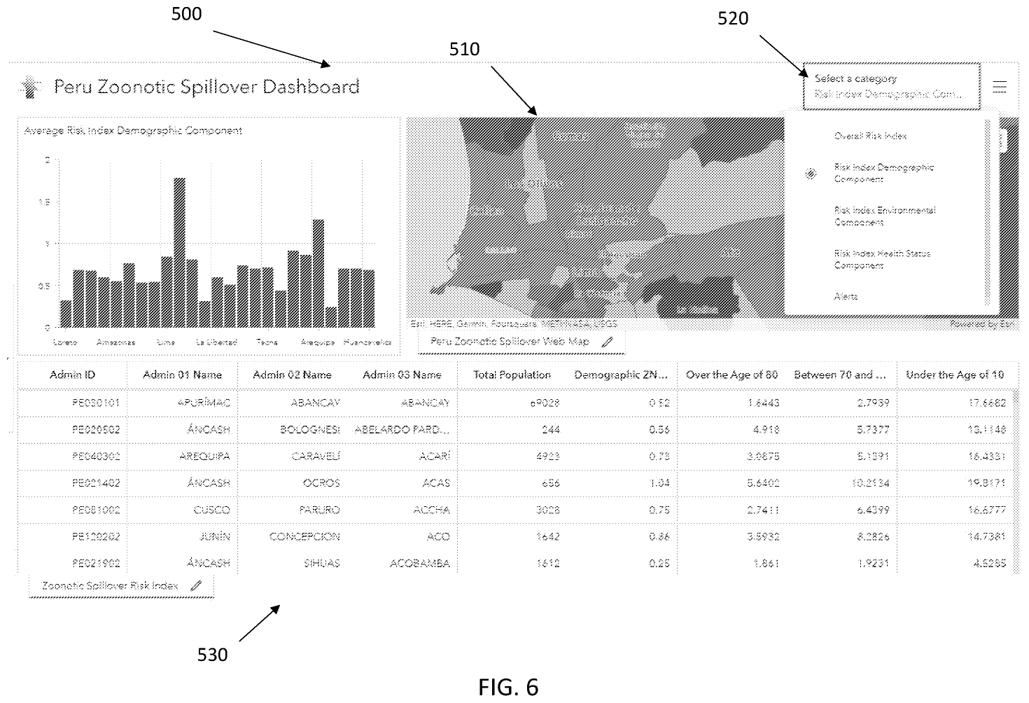

In some embodiments, as illustrated in FIG. 6, by selecting the “Risk Index Demographic Component” option from the menu 520, the thematic map 510 changes to show just the impact of the demographic components and may include a bar chart of risk from the demographic components by Administrative I Unit (i.e., the equivalent of states in the U.S.). The table changes as well in the information display area 530 to show the value of the specific data inputs of the demographic components by Administrative III Unit.

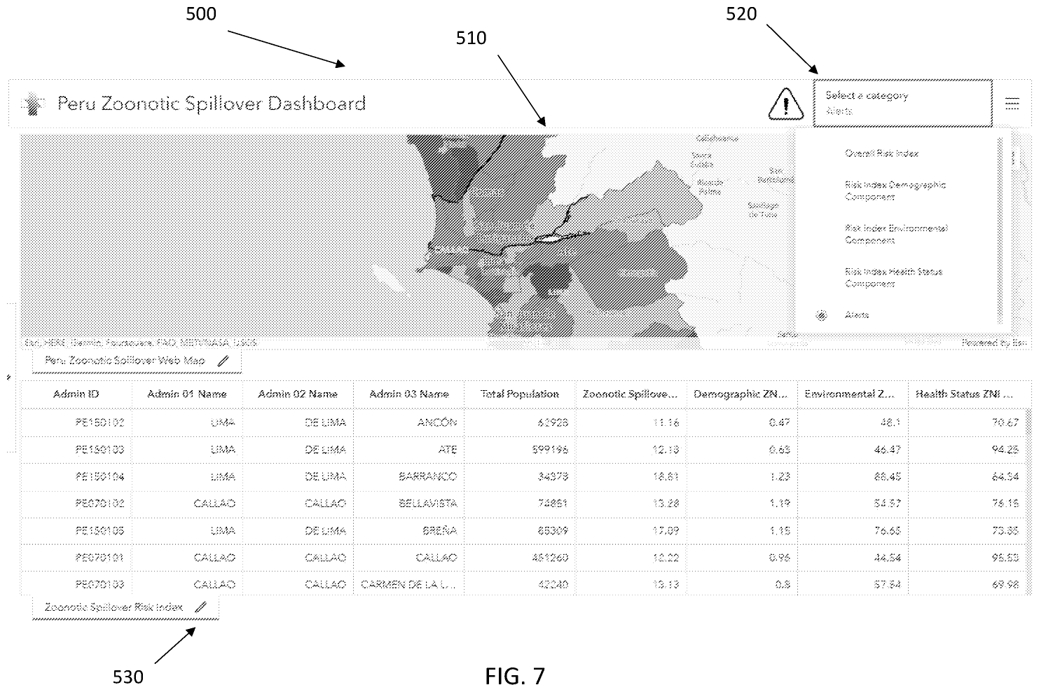

In some embodiments, as illustrated in FIG. 7, by selecting the “Alerts” option from the menu 520, the localities of interest having the highest Zoonotic Spill-Over Risk Index may be provided in the information display area 530. In an embodiment, the alerts may be continuously provided such that the highest alerts are visualized.

As such, such change in visual appearance of the dashboard enables a deeper analysis of Zoonotic Spill-Over Risk from the subset of risk factors. Similarly, the dashboard 500 may illustrate, based on user selection, details on other groupings or categorizes of zoonotic spill-over risk factors. Such change in visual appearance may occur automatically, e.g., based on the zoonotic spill-over risk value and change thereon or alerts, such that the Administrative Ill units having the highest/greatest risk of the zoonotic spill-over event automatically appears and/or changed thereto.

Moreover, in some embodiments, the changes in appearance of the dashboard 500 may occur dynamically. For example, in some embodiments, the dashboard 500 for the Zoonotic Spill-Over Risk Index may automatically change its display based on key changes to an input. For instance, changes to the extent or zone of influence of disease-carrying insects may impact zoonotic spill-over risk and determination thereon. For example, Peru is often hit with floods, such as in its Rimac River basin, which may be used as an input or indication for a dynamic change, e.g., during a raining season. That is, should, for example, a flood occur in Peru's Rimac River basin effecting the natural habitat of such insects, the dashboard may immediately change its display to focus the map on the geographic regions impacted and show the data on risk factors relevant to that region. Additional alert features such as an audio sound and email or text messages may also be possible to alert the decision maker(s).

As discussed above, the methods and system for the zoonotic spill-over risk analytics and/or prediction includes at least the following:

Identify the problem and its specific driving factors.

Identify the magnitude of the problem, relative to prediction of a zoonotic spill-over event

Leverage the decision tree to design an intervention specific to the identified problem and underlying ecological, environmental, human migration, animal and insect migration, land use change, and climate change related factors.

Target the interventions to the specific problem.

The zoonotic spill-over risk analytics and/or prediction process or system as described herein are configured to determine factors, e.g., ecological, environmental, human migration, animal and insect migration, land use change, and climate change related factors, for predicting the risks of zoonotic spillover events occurring, for example, in different geographical locations, e.g., localities of interest, and/or determining when a zoonotic spillover event has occurred. As such, the systems and methods as described herein are directed to developing a more holistic understanding of the risk and risk factors for the prediction of zoonotic spillover events using advanced machine learning models that are trained on diverse datasets. The advanced machine learning models may be trained on a unique, fit-for-purpose infrastructure with adjustable components such that the overall infrastructure is specifically designed for the ingesting and analysis of the identified diverse datasets that are specifically relevant to zoonotic spillover threats and/or risk for the localities of interest. The infrastructure may also be configured to provide for the easier and quicker visualization and consumption of the risk of a zoonotic spill-over event occurring, threat identification of specific risk factors, as well as communication of both the specific risk factors (for a locality of interest) and/or action/response plans to mitigate the risk level for the zoonotic spill-over threat.

### Use Cases

The identification of the specific risk factor (human migration, human mobility, animal migration, animal habitat loss, deforestation, environmental factors (e.g., soil moisture loss), climate change (e.g., rising temperatures), etc.) that most contribute to the overall risk of zoonotic spill-over for a given location/region may be identified.

For example, in an embodiment, as applied globally, the identification of those regions that have a greater relative risk of a zoonotic spill-over event occurring versus other regions can inform where, from a global perspective, disease surveillance efforts may be directed. For instance—while monitoring disease progression and disease spill-over between animal/insect and human globally may be untenable, close monitoring of the, say, 2-5 highest risk regions for such disease spill-over may be possible, for instance through the targeting of satellites and/or use of drone aircraft to capture Earth Observation data of the high risk regions. As such, the allocating of resources—and reallocating resources as on-the-ground risk conditions change—to monitor high risk regions may speed the discovery of a zoonotic disease spill-over event, speed the launch of mitigation and response measures to limit the disease spread and more effectively treat the affected.

In an embodiment, as applied locally, the identification of the specific risk factors contributing to zoonotic spill-over risk in a given location/region may inform local government officials, planners (e.g., city planners), economic development officials, health officials, public health decision makers, etc., on specific measures to address and lower the risk of zoonotic spill-over. For instance, a particular nation/region may direct economic development (e.g., urbanization) in areas that minimize disruption to potential-disease carrying animal/insect populations, or may take other measures to limit overlap and interaction between potential-disease carrying animal/insect populations and human communities/populations.

As such, the embodiments as discussed herein for providing zoonotic spill-over risk analytics and/or prediction may be used to provide the following:

The identification of the relative risk of a zoonotic spill-over event occurring in a given location/region as high, medium, or low (if on a 3-point scale) or identification of the relative risk of a zoonotic spill-over event in a location/region based on the relativity position of the location/region on a spectrum of risk between low and high.

Comparison of relative risk of zoonotic spill-over between two locations/regions in terms of which location/region has a higher risk of zoonotic spill-over.

The identification of the specific risk factor or factors (human migration, human mobility, animal migration, animal habitat loss, deforestation, environmental factors (e.g., soil moisture loss), Climate change (e.g., rising temperatures), etc., most contributing to the overall risk of zoonotic spill-over for a given location/region.

The identification of the specific risk factor or factors (human migration, human mobility, animal migration, animal habitat loss, deforestation, environmental factors (e.g., soil moisture loss), climate change (e.g., rising temperatures), etc., least contributing to the overall risk of zoonotic spill-over for a given location/region.

It is to be understood that the disclosed and other solutions, examples, embodiments, modules, events, functions, and the functional operations described in this document may be implemented in digital electronic circuitry, or in computer software, firmware, or hardware, including the structures disclosed in this document and their structural equivalents, or in combinations of one or more of them. The disclosed and other embodiments may be implemented as one or more computer program products, i.e., one or more modules of computer program instructions encoded on a computer readable medium for execution by, or to control the operation of, data processing apparatus. The computer readable medium may be a machine-readable storage device, a machine-readable storage substrate, a memory device, a composition of matter effecting a machine-readable propagated signal, or a combination of one or more them. The term “data processing apparatus” encompasses all apparatus, devices, and machines for processing data, including by way of example a programmable processor, a computer, or multiple processors or computers. The apparatus may include, in addition to hardware, code that creates an execution environment for the computer program in question, e.g., code that constitutes processor firmware, a protocol stack, a database management system, an operating system, or a combination of one or more of them.

A computer program (also known as a program, software, software application, script, or code) may be written in any form of programming language, including compiled or interpreted languages, and it may be deployed in any form, including as a stand-alone program or as a module, component, subroutine, or other unit suitable for use in a computing environment. A computer program does not necessarily correspond to a file in a file system. A program may be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program may be deployed to be executed on one computer or on multiple computers that are located at one site or distributed across multiple sites and interconnected by a communication network.

One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments. Additionally, while the above has been discussed with respect to methods and systems, it is appreciated that the methods may be stored on non-transitory computer-readable medium having computer-readable instructions, which when executed by a processor, performs the above steps of operation.

Different features, variations and multiple different embodiments have been shown and described with various details. What has been described in this application at times in terms of specific embodiments is done for illustrative purposes only and without the intent to limit or suggest that what has been conceived is only one particular embodiment or specific embodiments. It is to be understood that this disclosure is not limited to any single specific embodiments or enumerated variations. Many modifications, variations and other embodiments will come to mind of those skilled in the art, and which are intended to be and are in fact covered by both this disclosure. It is indeed intended that the scope of this disclosure should be determined by a proper legal interpretation and construction of the disclosure, including equivalents, as understood by those of skill in the art relying upon the complete disclosure present at the time of filing.

The herein described subject matter sometimes illustrates different components contained within, or connected with, different other components. It is to be understood that such depicted architectures are merely examples, and that in fact many other architectures may be implemented which achieve the same functionality. In a conceptual sense, any arrangement of components to achieve the same functionality is effectively “associated” such that the desired functionality is achieved. Hence, any two components herein combined to achieve a particular functionality may be seen as “associated with” each other such that the desired functionality is achieved, irrespective of architectures or intermedial components. Likewise, any two components so associated may also be viewed as being “operably connected”, or “operably coupled”, to each other to achieve the desired functionality, and any two components capable of being so associated may also be viewed as being “operably couplable”, to each other to achieve the desired functionality. Specific examples of operably couplable include but are not limited to physically mateable and/or physically interacting components and/or wirelessly interactable and/or wirelessly interacting components and/or logically interacting and/or logically interactable components.

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting.

What is claimed is:

1. A method of providing zoonotic spill-over risk analytics and/or prediction, comprising:
obtaining a first set of ecological, environmental, human migration, animal and insect migration, land use change, and climate change related data from at least one data source for at least one first locality of interest;
removing source identification information from the first set of ecological, environmental, human migration, animal and insect migration, land use change, and climate change related data;
obtaining data on infection or disease incidence for the at least one locality of interest;
training a model to:
identify an impact of ecological, environmental, human migration, animal and insect migration, land use change, and climate change related variables on the infection or disease incidence by determining which of the ecological, environmental, human migration, animal and insect migration, land use change, and climate change related factors impact the infection or disease incidence for the at least one locality of interest, and
based on the ecological, environmental, human migration, animal and insect migration, land use change, and climate change related variables having a highest impact on the infection or disease incidence, determine a zoonotic spill-over risk index for predicting a spill-over event for the at least one locality of interest;
implementing the model with a second set of ecological, environmental, human migration, animal and insect migration, land use change, and climate change related data from the at least one data source for at least a second locality of interest to determine the zoonotic spill-over risk index for the at least second locality of interest; and
displaying at least one of the zoonotic spill-over risk index for the at least second locality of interest and the ecological, environmental, human migration, animal and insect migration, land use change, and climate change related variables having the highest impact on the zoonotic spill-over risk index for the at least second locality of interest.

2. The method of claim 1, further comprising:
wherein the at least one first locality of interest is the same as the at least second locality of interest.

3. The method of claim 1, further comprising:
wherein the at least one first locality of interest is different than the at least second locality of interest.

4. The method of claim 1, further comprising:
removing any geospatial data from the first set of ecological, environmental, human migration, animal and insect migration, land use change, and climate change related data.

5. The method of claim 4, further comprising:
reattaching the geospatial data to the zoonotic spill-over risk index; and
the displaying includes providing a mapping and/or visual geospatial representation of the zoonotic spill-over risk index at different geographical levels for the at least second locality of interest.

6. The method of claim 1, wherein the obtaining the first set of ecological, environmental, human migration, animal and insect migration, land use change, and climate change related data is through at least one of an application programming interface, webscraping, direct download from a source site, and cloud objects.

7. The method of claim 1, wherein the identifying the impact of ecological, environmental, human migration, animal and insect migration, land use change, and climate change related factors on the infection or disease incidence includes transforming the first set of ecological, environmental, human migration, animal and insect migration, land use change, and climate change related data into a risk signal associated with the first set of ecological, environmental, human migration, animal and insect migration, land use change, and climate change related data.

8. The method of claim 1, wherein the determining which of the ecological, environmental, human migration, animal and insect migration, land use change, and climate change related factors have the highest impact includes leveraging a bi-variate cluster analysis to identify specific ecological, environmental, human migration, animal and insect migration, land use change, and climate change related variables that impact the infection or disease incidence.

9. The method of claim 1, wherein the obtaining the first set of ecological, environmental, human migration, animal and insect migration, land use change, and climate change related data further includes processing image data into numerical data.

10. The method of claim 9, wherein the processing of the image data includes converting the set of ecological, environmental, human migration, animal and insect migration, land use change, and climate change related data into tabular form after removing any of the geospatial data from the image data.

11. The method of claim 1, further comprising:
displaying a plurality of localities of interest having the highest zoonotic spill-over risk index and/or the ecological, environmental, human migration, animal and insect migration, land use change, and climate change related variables associated with the highest impact; and
when the plurality of localities of interest change based on any change to the localities of interest having the highest zoonotic spill-over risk index, displaying any updated localities of interest having a new highest zoonotic spill-over risk index and new ecological, environmental, human migration, animal and insect migration, land use change, and climate change related variables associated with the highest impact associated with the new highest zoonotic spill-over risk index.

12. A non-transitory computer-readable medium having computer-readable instructions that, if executed by a computing device, cause the computing device to perform operations comprising the method of claim 1.

13. A system for providing zoonotic spill-over risk analytics and/or prediction comprising:
a plurality of data sources having ecological, environmental, human migration, animal and insect migration, land use change, and climate change related data;
a cloud-based system configured to obtain a first set of the ecological, environmental, human migration, animal and insect migration, land use change, and climate change related data from the plurality of data sources, wherein the cloud-based system includes machine-learning algorithms and is configured to:
remove source identification information from the first set of ecological, environmental, human migration, animal and insect migration, land use change, and climate change related data,
obtaining data on infection or disease incidence for the at least one locality of interest,
identify an impact of ecological, environmental, human migration, animal and insect migration, land use change, and climate change related variables on the infection or disease incidence by determining which of the ecological, environmental, human migration, animal and insect migration, land use change, and climate change related factors impact the infection or disease incidence for the at least one locality of interest,
based on the ecological, environmental, human migration, animal and insect migration, land use change, and climate change related variables having a highest impact on the infection or disease incidence, determine a zoonotic spill-over risk index for predicting a spill-over event for the at least one locality of interest,
implement the model with a second set of ecological, environmental, human migration, animal and insect migration, land use change, and climate change related data from the at least one data source for at least second locality of interest to determine the zoonotic spill-over risk index for the at least second locality of interest; and
transmit at least one of the zoonotic spill-over risk index for the at least second locality of interest and the ecological, environmental, human migration, animal and insect migration, land use change, and climate change related variables having the highest impact on the zoonotic spill-over risk index for the at least second locality of interest.

14. The system of claim 13, wherein the cloud-based system is further configured to:
remove any geospatial data from the first set of ecological, environmental, human migration, animal and insect migration, land use change, and climate change related data; and
reattach the geospatial data to the zoonotic spill-over risk index before transmitting the zoonotic spill-over risk index to the cloud-based server.

15. The system of claim 14, further comprising a display for providing a mapping and/or visual geospatial representation of the zoonotic spill-over risk index at different geographical levels for the at least second locality of interest.

16. The system of claim 13, wherein the identifying the impact of ecological, environmental, human migration, animal and insect migration, land use change, and climate change related factors on the infection or disease incidence includes transforming the first set of ecological, environmental, human migration, animal and insect migration, land use change, and climate change related data into a risk signal associated with the first set of ecological, environmental, human migration, animal and insect migration, land use change, and climate change related data.

17. The system of claim 13, wherein the obtaining the first set of ecological, environmental, human migration, animal and insect migration, land use change, and climate change related data further includes processing image data into numerical data.
